(19) United States
(12) Patent Application Publication
(10) Pub. No.: US 2016/0045311 A1
(43) Pub. Date: Feb. 18, 2016

(54) PROSTHETIC IMPLANT DELIVERY DEVICE

(71) Applicant: Direct Flow Medical, Inc., Santa Rosa, CA (US)

(72) Inventors: Alex Phillip McCann, Santa Rosa, CA (US); Terry W. Daniels, Santa Rosa, CA (US); David Eron Flory, The Sea Ranch, CA (US); Kevin C. Robin, Santa Rosa, CA (US)

(21) Appl. No.: 14/826,112

(22) Filed: Aug. 13, 2015

Related U.S. Application Data
(60) Provisional application No. 62/038,066, filed on Aug. 15, 2014.

(51) Int. Cl. A61F 2/24; A61M 25/01; A61B 19/00; A61M 25/00

(52) U.S. Cl. CPC A61F 2/2436; A61F 2/2412; A61F 2/2418; A61M 25/0097; A61M 25/0113; A61B 19/30; A61F 2250/0003

(57) ABSTRACT

The disclosure includes an arrangement for a handle for a catheter system that includes a first member and a second member. The handle can include a screw member positioned within the handle and configured for rotation about an axis. The screw member can include an internal thread. A carriage can be positioned within the screw member. The carriage can engage the internal thread and can be coupled to the first member. An alignment member can extend within the screw member to limit rotation of the carriage about the axis as the screw member is rotated. Rotation of the screw member in a first direction about the axis can cause the carriage to move in a first longitudinal direction within the screw member causing the first member to move in the first longitudinal direction relative to the handle.

### INCORPORATION BY REFERENCE RELATED APPLICATIONS

This application claims the benefit of U.S. Provisional Application No. 62/038,066, filed Aug. 15, 2014 the entirety of which is hereby incorporated by reference herein.

### BACKGROUND

1. Field

The present invention relates to medical methods and devices, and, more specifically, to methods and devices for percutaneously implanting a valve.

2. Description of the Related Art

The circulatory system is a closed loop bed of arterial and venous vessels supplying oxygen and nutrients to the body extremities through capillary beds. The driver of the system is the heart providing correct pressures to the circulatory system and regulating flow volumes as the body demands. Deoxygenated blood enters heart first through the right atrium and is allowed to the right ventricle through the tricuspid valve. Once in the right ventricle, the heart delivers this blood through the pulmonary valve and to the lungs for a gaseous exchange of oxygen. The circulatory pressures carry this blood back to the heart via the pulmonary veins and into the left atrium. Filling of the left atrium occurs as the mitral valve opens allowing blood to be drawn into the left ventricle for expulsion through the aortic valve and on to the body extremities. When the heart fails to continuously produce normal flow and pressures, a disease commonly referred to as heart failure occurs.

The four valves of the heart (i.e., the tricuspid, the pulmonary valve, the mitral valve and the aortic valve) function to ensure that blood flows in only one direction through the heart. The valves are made of thin flaps of tissue that open and close as the heart contracts. Valvular heart disease is any disease process involving one or more of the valves of the heart. For example, disease and age can cause the tissue of a heart valve to thicken and harden, which can case the valve to fail to open properly and interfere with blood flow. This thickening process is often called stenosis. A heart valve can also become weakened or stretched such it no longer closes properly, which can cause blood leak back through the valve. This leakage through the valve is often called regurgitation. Problems with a heart valve can increase the amount of work performed by the heart. The increase in work can cause the heart muscle to enlarge or thicken to make up for the extra workload.

The standard treatment for replacing an improperly working valve is to replace it. Traditionally, valve replacement has been accomplished via an open surgical procedure. More recently, transcatheter valve replacement has been attempted via percutaneous method such as a catheterization or delivery mechanism utilizing the vasculature pathways. Open surgical procedures often include the sewing of a new valve to the existing tissue structure for securement. Access to these sites generally include a thoracotomy or a sternotomy for the patient and include a great deal of recovery time. Such open-heart surgical procedures can include placing the patient on heart bypass to continue blood flow to vital organs such as the brain during the surgery. Although open heart surgical valve repair and replacement can successfully treat many patients with valvular insufficiency, techniques currently in use are attended by significant morbidity and mortality due to the inherent invasiveness of open heart surgery.

According to recent estimates, more than 79,000 patients are diagnosed with aortic and mitral valve disease in U.S. hospitals each year. More than 49,000 mitral valve or aortic valve replacement procedures are performed annually in the U.S., along with a significant number of heart valve repair procedures. Since surgical techniques are highly invasive, the need for a less invasive method of heart valve replacement has long been recognized. As noted above, transcatheter heart valve systems have recently been developed in which heart valves are delivered through the heart by an intravascular catheter. Such transcatheter heart valves have the potential to reduce the anticipated mortality and morbidity rates associated with traditional surgical valve surgery particularly among patients of advanced age and/or with comorbidities. However, a need remains for improvements over the basic concept of transcatheter heart valve replacement. For example, current transcatheter valve replacement can sometimes result in vascular complications such as aortic dissection, access site or access related vascular and/or distal embolization from a vascular source. One method for reducing such complications is to reduce ratio of the diameter of the delivery device for the heart valve.

### SUMMARY

An embodiment comprises a delivery system for delivering a cardiovascular prosthetic implant. The delivery system can include a delivery catheter comprising an outer sheath with a proximal end portion and an inner sheath extending at least partially through the outer sheath. The inner sheath can have a proximal end portion. A handle can be positioned at a proximal end portion of the delivery catheter. A screw member can be positioned at least partially within the handle. The screw member can be configured for rotation about an axis within the handle. The screw member can include an internal thread. A carriage can be positioned within the screw member and can engage the internal thread. The carriage is coupled to the proximal end portion of the outer sheath. An alignment member is positioned within the screw member. The alignment member contacts the carriage to limit rotation of the carriage about the axis as the screw member is rotated. Rotation of the screw member in a first direction about the axis causes the carriage to move in a first longitudinal direction within the screw member causing the outer sheath to move in the first longitudinal direction relative to the handle.

Another embodiment comprises a method of positioning a prosthetic implant within a heart. The method can include advancing a delivery catheter comprising a prosthetic valve positioned within an outer sheath into a patient's vascular system; translumenally advancing the prosthetic valve to a position proximate a native valve of the heart; and deploying the prosthetic valve by retracting the outer sheath by rotating a screw member positioned within a handle of the delivery catheter to cause a carriage coupled to the outer sheath and positioned within the screw member to linearly retract within the screw cylinder as the screw member is rotated.

Another embodiment comprises a handle for a catheter system that includes a first member and a second member. The handle can include a screw member positioned within the handle and configured for rotation about an axis. The screw member includes an internal thread. A carriage is positioned within the screw member. The carriage engages the internal thread and is coupled to the first member. An alignment member extends within the screw member to limit rotation of the carriage about the axis as the screw member is rotated. Rotation of the screw member in a first direction about the axis causes the carriage to move in a first longitudinal direction within the screw member causing the first member to move in the first longitudinal direction relative to the handle.

Another embodiment comprises a method of retracting and outer sheath relative to an inner sheath of a catheter that can include rotating a screw member positioned within a handle of the delivery catheter to cause a carriage coupled to the outer sheath and positioned within the screw member to linearly retract within the screw cylinder as the screw member is rotated.

Further features arrangements, embodiments, and advantages of the present invention will become apparent from the detailed description of the embodiments which follows, when considered together with the attached drawings and claims.

### BRIEF DESCRIPTION OF THE DRAWINGS

FIG. 1 is a cross-sectional schematic view of a heart and its major blood vessels.

FIG. 2A is a partial cut-away view a left ventricle and aortic with an prosthetic aortic valve implant according to one embodiment.

FIG. 2B is a side view of the implant of FIG. 2A positioned across a native aortic valve.

FIG. 3A is a front perspective view of the implant of FIG. 2B.

FIG. 3B is a front perspective view of an inflatable support structure of the implant of FIG. 3A.

FIG. 3C is a cross-sectional side view of the implant of FIG. 3A.

FIG. 3D is an enlarged cross-sectional view of an upper portion of FIG. 3C.

FIG. 4 is a cross-sectional view of the connection port and the inflation valve in the implant of FIG. 3B.

FIG. 5A is a side perspective view of a deployment catheter with retracted implant.

FIG. 5B is a side perspective view of the deployment catheter of FIG. 5A with the implant outside of the outer sheath jacket.

FIG. 5C is a side perspective view of the position-and-fill lumen (PFL), which is a component of the deployment catheter of FIGS. 5A and 5B.

FIG. 6 is a cross-sectional view taken through line A-A of FIG. 5B.

FIG. 7 is a side perspective view of a loading tool base.

FIG. 8A is a side perspective view of an introduced catheter deployment catheter with retracted implant.

FIG. 8B is a side perspective view of the introducer catheter and deployment catheter of FIG. 8A with the implant outside of the outer sheath jacket.

FIG. 8C is a side perspective view of the position-and-fill lumen (PFL), which is a component of the deployment catheter of FIGS. 8A and 8B.

FIG. 9 is a side view of the introducer catheter of FIGS. 8A-8C.

FIG. 10A is a side view of the deployment catheter of FIGS. 8A-8C.

FIG. 10B is an exploded view of a seal assembly.

FIG. 11A illustrates a step of partially deploying and positioning an artificial valve implant.

FIG. 11B illustrates a second step of partially deploying and positioning an artificial valve implant.

FIG. 11C illustrates a third step of partially deploying and positioning an artificial valve implant.

FIG. 12A illustrates a step deploying, testing and repositioning an artificial valve implant.

FIG. 12B illustrates a step deploying, testing and repositioning an artificial valve implant.

FIG. 12C illustrates a step deploying, testing and repositioning an artificial valve implant.

FIG. 12D illustrates a step deploying, testing and repositioning an artificial valve implant.

FIG. 12E illustrates a step deploying, testing and repositioning an artificial valve implant.

FIG. 13 illustrates a side view of another embodiment of a deployment system.

FIG. 14 illustrates a side view of another embodiment of a deployment system.

FIG. 15A is a side schematic illustration of another embodiment of a deployment system.

FIG. 15B is a side cross-sectional schematic illustration of the system of FIG. 15A.

FIG. 16A is a top perspective view of another embodiment of a deployment system including an outer sheath, a knob and a handle.

FIG. 16B is a top perspective view of the deployment system of FIG. 15 with the outer sheath omitted.

FIG. 17 is a bottom perspective view of the deployment system of FIG. 16B.

FIG. 18 is a bottom perspective view of the deployment system of FIG. 17 with a top cover of the handle removed.

FIG. 19 is a side perspective view of a front portion the deployment system of FIG. 17 with a bottom cover of the handle removed.

FIG. 20A is a top view of the deployment system of FIG. 16B with a top cover of the handle removed.

FIG. 20B is a closer view of a portion of FIG. 20A.

FIG. 21 is an exploded side perspective view of the knob, a screw member, a carriage, a track member, and a locking mechanism of the deployment system of FIG. 16B.

FIG. 22 is an exploded side perspective view of some of the components of the deployment system of FIG. 16B.

FIG. 23 is an exploded side perspective view of some of the components of the deployment system of FIG. 16B.

FIGS. 24A and 24B illustrate movement of a carriage within the handle of the deployment system of FIG. 15 with the screw member and knob omitted.

FIG. 25A is a side view of the screw member and a portion of the handle with a top cover removed.

FIG. 25B is a side view of the carriage positioned within the screw member.

FIG. 25C is a front view of the carriage positioned within the screw member.

FIG. 26A is a side view of the screw member positioned within the knob.

FIG. 26B is a side view of the screw member positioned within the knob in along a different cross-section.

FIG. 26C is a review view of the carriage screw member positioned within the knob.

FIG. 27 is a rear side perspective view of the carriage.

FIG. 28 is a front perspective view of the carriage.

FIG. 29 is a front perspective view of the locking mechanism in a locked position.

FIG. 30 is a front perspective view of the locking mechanism in an unlocked position.

FIG. 31 is an exploded front side view of the locking mechanism of FIG. 29.

### DETAILED DESCRIPTION

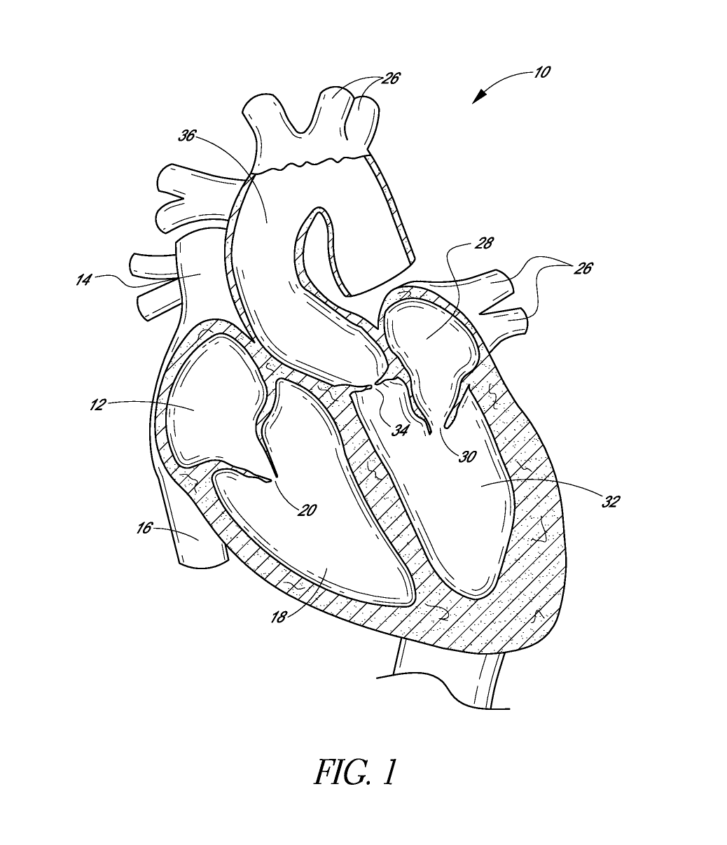

FIG. 1 is a schematic cross-sectional illustration of the anatomical structure and major blood vessels of a heart 10. Deoxygenated blood is delivered to the right atrium 12 of the heart 10 by the superior and inferior vena cava 14, 16. Blood in the right atrium 12 is allowed into the right ventricle 18 through the tricuspid valve 20. Once in the right ventricle 18, the heart 10 delivers this blood through the pulmonary valve 22 to the pulmonary arteries 24 and to the lungs for a gaseous exchange of oxygen. The circulatory pressures carry this blood back to the heart via the pulmonary veins 26 and into the left atrium 28. Filling of the left atrium 28 occurs as the mitral valve 30 opens allowing blood to be drawn into the left ventricle 32 for expulsion through the aortic valve 34 and on to the body extremities through the aorta 36. When the heart 10 fails to continuously produce normal flow and pressures, a disease commonly referred to as heart failure occurs.

One cause of heart failure is failure or malfunction of one or more of the valves of the heart 10. For example, the aortic valve 34 can malfunction for several reasons. For example, the aortic valve 34 may be abnormal from birth (e.g., bicuspid, calcification, congenital aortic valve disease), or it could become diseased with age (e.g., acquired aortic valve disease). In such situations, it can be desirable to replace the abnormal or diseased valve 34.

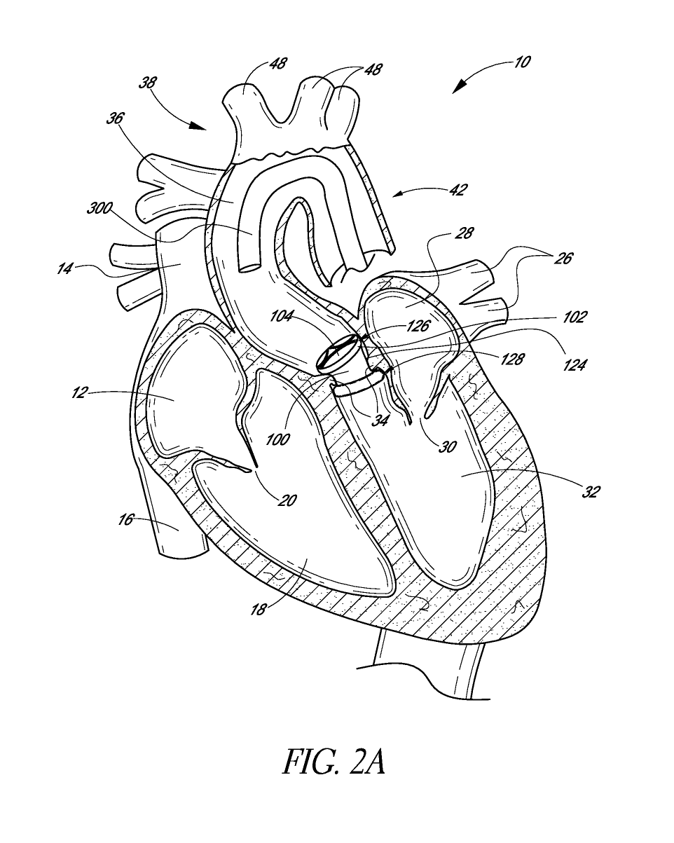

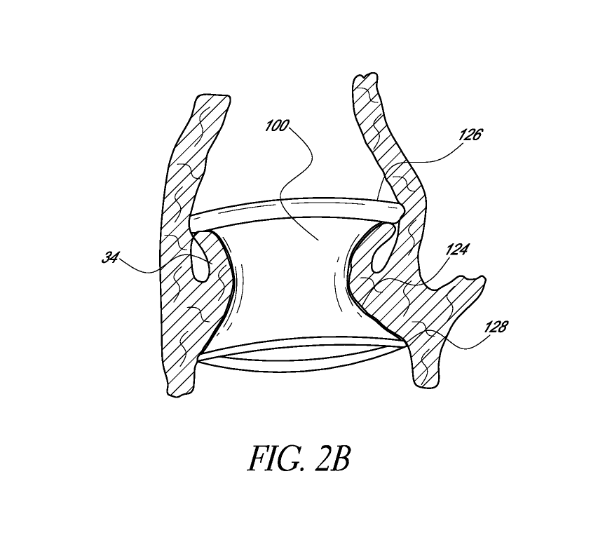

FIG. 2 is a schematic illustration of the left ventricle 32, which delivers blood to the aorta 36 through the aortic valve 34. The aorta 36 comprises (i) the ascending aorta 38, which arises from the left ventricle 32 of the heart 10, (ii) the aortic arch 10, which arches from the ascending aorta 38 and (iii) the descending aorta 42 which descends from the aortic arch 40 towards the abdominal aorta (not shown). Also shown are the principal branches of the aorta 14, which include the innomate artery 44 that immediately divides into the right carotid artery (not shown) and the right subclavian artery (not shown), the left carotid 46 and the subclavian artery 48.

### Inflatable Prosthetic Aortic Valve Implant

With continued reference to FIG. 2A, a cardiovascular prosthetic implant 800 in accordance with one embodiment is shown spanning the native abnormal or diseased aortic valve 34. The implant 800 and various modified embodiments thereof will be described in detail below. As will be explained in more detail below, the implant 800 can be delivered minimally invasively using an intravascular delivery catheter 900 or trans apical approach with a trocar. Further details, additional embodiments of and/or modifications of the implant or delivery system can be found in U.S. Pat. Nos. 7,641,686, 8,012,201 and U.S. Publication Nos. 2007/0005133; 2009/0088836 and 2012/0016468, the entirety of these patents and publications are hereby incorporated by reference herein in their entirety.

The description below will be primarily in the context of replacing or repairing an abnormal or diseased aortic valve 34. However, various features and aspects of methods and structures disclosed herein are applicable to replacing or repairing the mitral 30, pulmonary 22 and/or tricuspid 20 valves of the heart 10 as those of skill in the art will appreciate in light of the disclosure herein. In addition, those of skill in the art will also recognize that various features and aspects of the methods and structures disclosed herein can be used in other parts of the body that include valves or can benefit from the addition of a valve, such as, for example, the esophagus, stomach, ureter and/or vesicle, biliary ducts, the lymphatic system and in the intestines.

In addition, various components of the implant and its delivery system will be described with reference to coordinate system comprising “distal” and “proximal” directions. In this application, distal and proximal directions refer to the deployment system 900, which is used to deliver the implant 800 and advanced through the aorta 36 in a direction opposite to the normal direction of blood through the aorta 36. Thus, in general, distal means closer to the heart while proximal means further from the heart with respect to the circulatory system.

In some embodiments, the implant 800 can be a prosthetic aortic valve implant. With reference to FIG. 2B in the illustrated embodiment, the implant 800 can have a shape that can be viewed as a tubular member or hyperboloid shape where a waist 805 excludes the native valve 34 or vessel and proximally the proximal end 803 forms a hoop or ring to seal blood flow from re-entering the left ventricle 32. Distally, the distal end 804 can also form a hoop or ring to seal blood from forward flow through the outflow track. Between the two ends 803 and 804, a valve 104 can be mounted to the cuff or body 802 such that when inflated the implant 800 excludes the native valve 34 or extends over the former location of the native valve 34 and replaces its function. The distal end 804 can have an appropriate size and shape so that it does not interfere with the proper function of the mitral valve, but still secures the valve adequately. For example, there can be a notch, recess or cut out in the distal end 804 of the device to prevent mitral valve interference. The proximal end 803 can be designed to sit in the aortic root. In one arrangement, the proximal end 803 can be shaped in such a way that it maintains good apposition with the wall of the aortic root. This can prevent the device from migrating back into the ventricle 32. In some embodiments, the implant 800 can be configured such that it does not extend so high that it interferes with the coronary arteries.

Any number of additional inflatable rings or struts can be disposed between the proximal end 803 and distal end 804. The distal end 804 of the implant 800 can be positioned within the left ventricle 34 and can utilize the aortic root for axial stabilization as it may have a larger diameter than the aortic lumen. This arrangement may lessen the need for hooks, barbs or an interference fit to the vessel wall. Since the implant 800 can be placed without the aid of a dilatation balloon for radial expansion, the aortic valve 34 and vessel may not have any duration of obstruction and would provide the patient with more comfort and the physician more time to properly place the device accurately. Since in the illustrated arrangement, the implant 800 is not utilizing a support member with a single placement option as a plastically deformable or shaped memory metal stent does, the implant 800 can be movable and or removable if desired. This could be performed multiple times until the implant 800 is permanently disconnected from the delivery catheter 900 as will be explained in more detail below. In addition, as will be described below, the implant 800 can include features, which allow the implant 800 to be tested for proper function, sealing and sizing, before the catheter 900 is disconnected.

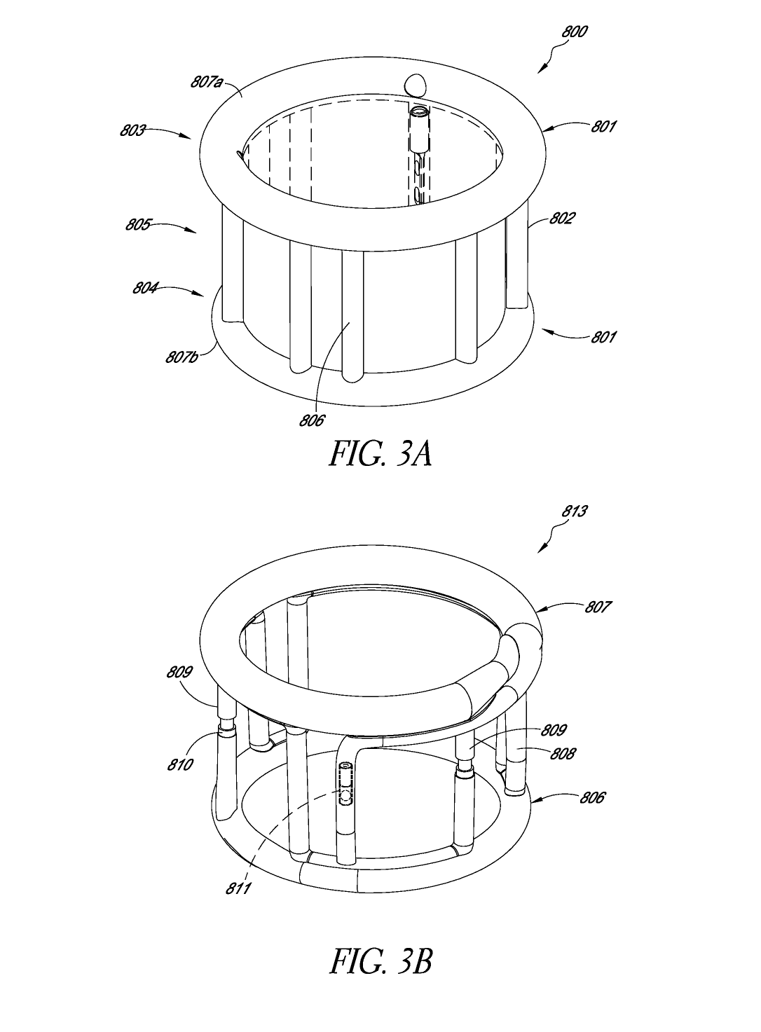

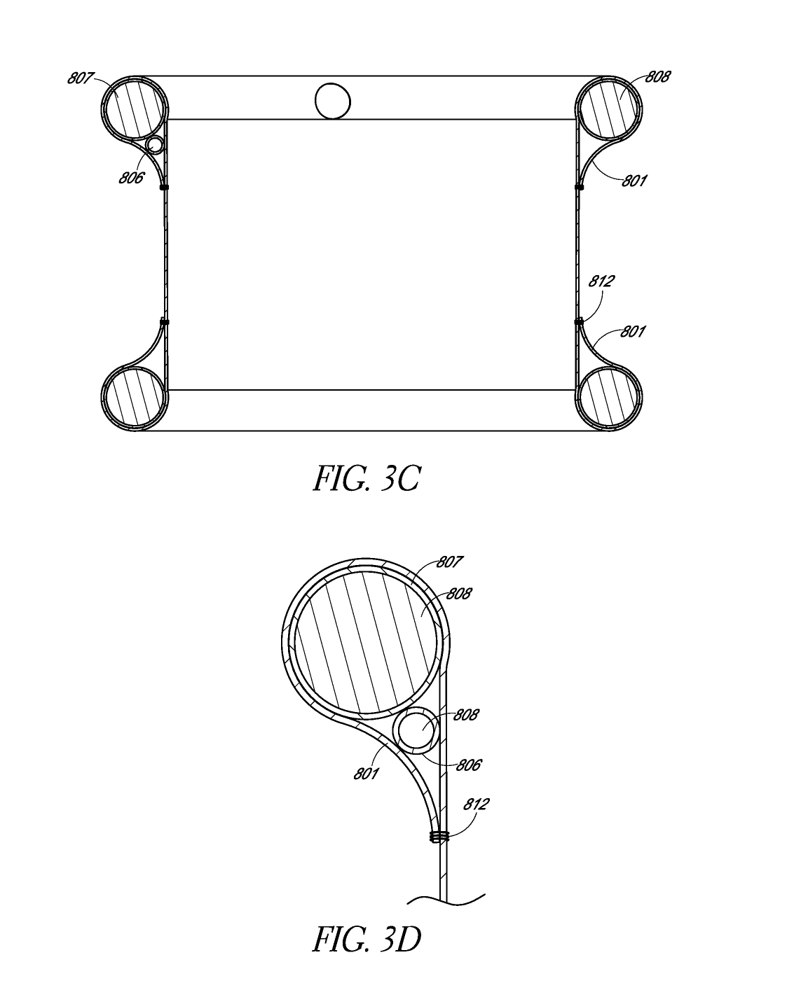

With reference to FIG. 3A, the implant 800 of the illustrated embodiment generally comprises the inflatable cuff or body 802, which is configured to support the valve 104 (see FIG. 2A) that is coupled to the cuff 802. In some embodiments, the valve 104 is a tissue valve. In some embodiments, the tissue valve has a thickness equal to or greater than about 0.011 inches. In another embodiment, the tissue valve has a thickness equal to or greater than about 0.018 inches. As will be explained in more detail below, the valve 104 can be configured to move in response to the hemodynamic movement of the blood pumped by the heart 10 between an “open” configuration where blood can throw the implant 800 in a first direction and a “closed” configuration whereby blood is prevented from back flowing through the valve 104 in a second direction.

In the illustrated embodiment, the cuff 802 can comprise a thin flexible tubular material such as a flexible fabric or thin membrane with little dimensional integrity. As will be explained in more detail below, the cuff 802 can be changed preferably, in situ, to a support structure to which other components (e.g., the valve 104) of the implant 800 can be secured and where tissue ingrowth can occur. Uninflated, the cuff 802 can be incapable of providing support. In one embodiment, the cuff 802 comprises Dacron, PTFE, ePTFE, TFE or polyester fabric as seen in conventional devices such as surgical stented or stent less valves and annuloplasty rings. The fabric thickness can range from about 0.002 inches to about 0.020 inches depending upon material selection and weave. Weave density may also be adjusted from a very tight weave to prevent blood from penetrating through the fabric to a looser weave to allow tissue to grow and surround the fabric completely. In certain embodiments, the fabric may have a linear mass density about 20 denier or lower.

With reference to FIGS. 3B-3D, in the illustrated embodiment, the implant 800 can include an inflatable structure 813 that is formed by one or more inflation channels 808. The inflatable channels 808 can be formed by a pair of distinct balloon rings or toroids (807a and 807b) and struts 806. In the illustrated embodiment, the implant 800 can include a proximal toroid 807a at the proximal end 803 of the cuff 802 and a distal toroid 807b at the distal end 804 of the cuff 802. The toroids 807 can be secured to the cuff 802 in any of a variety of manners. With reference to FIGS. 3C and 3D, in the illustrated embodiment, the toroids 807 can be secured within folds 801 formed at the proximal end 803 and the distal end 804 of the cuff 802. The folds 801, in turn, can be secured by sutures or stitches 812. When inflated, the implant 800 can be supported in part by series of struts 806 surrounding the cuff 802. In some embodiments, the struts 806 are configured so that the portions on the cuff run substantially perpendicular to the toroids. The struts can be sewn onto the cuff 802 or can be enclosed in lumens made from the cuff material and swan onto the cuff 802. The toroids 807 and the struts 806 together can form one or more inflatable channels 808 that can be inflated by air, liquid or inflation media.

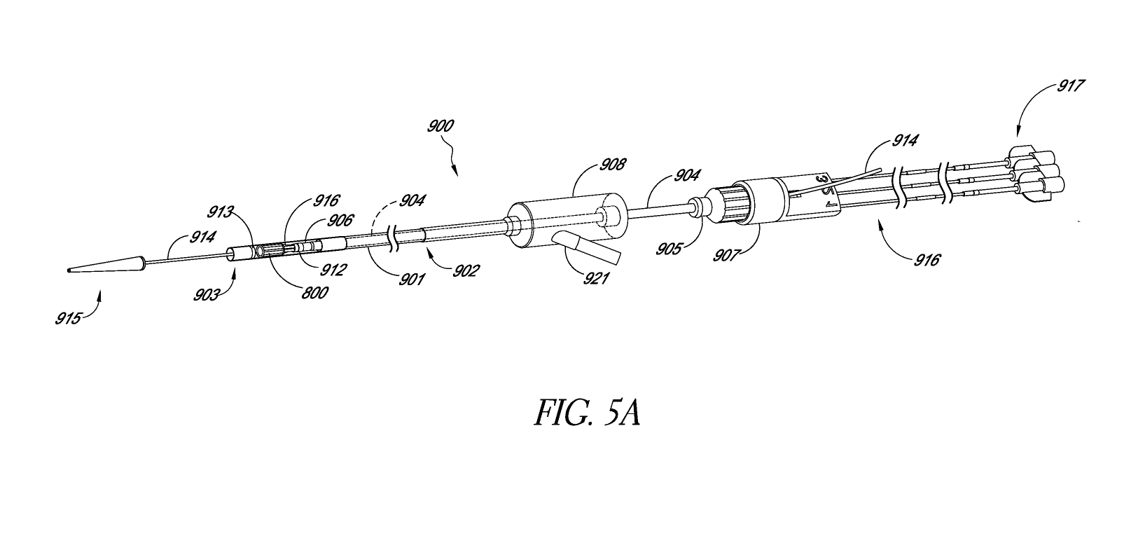

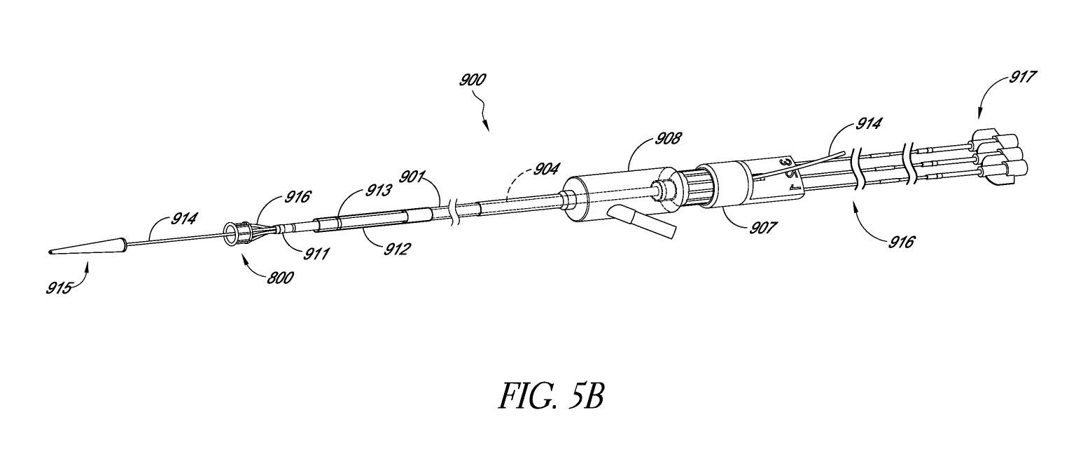

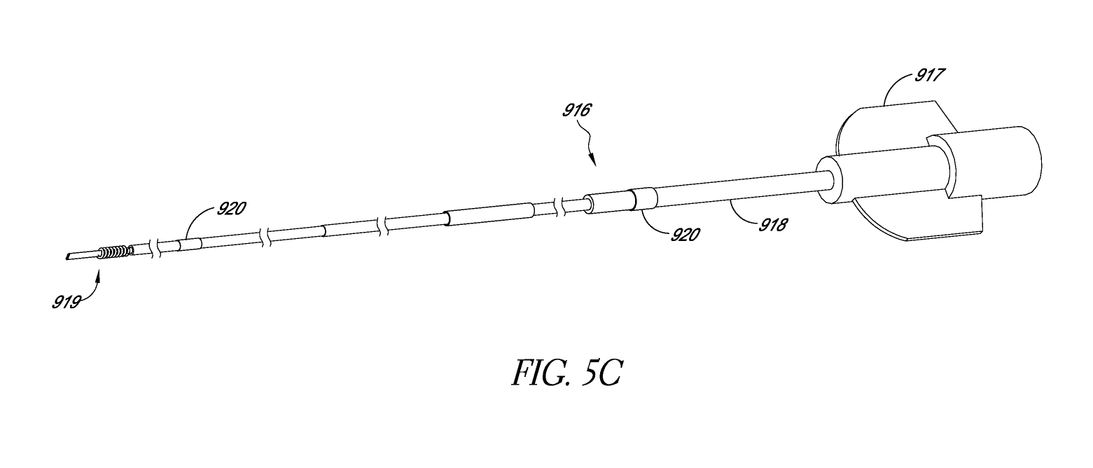

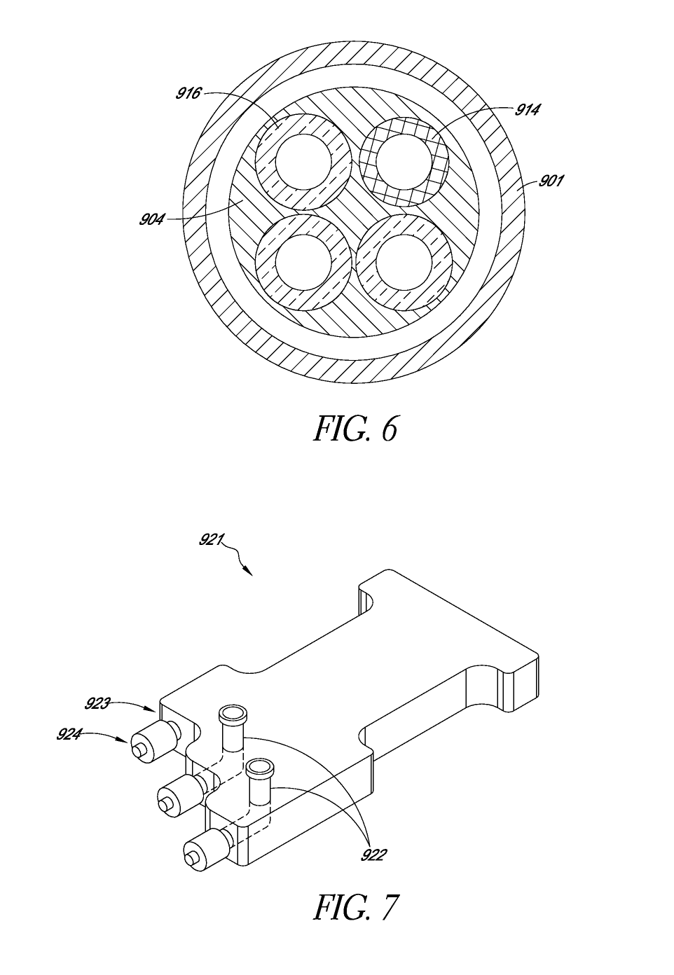

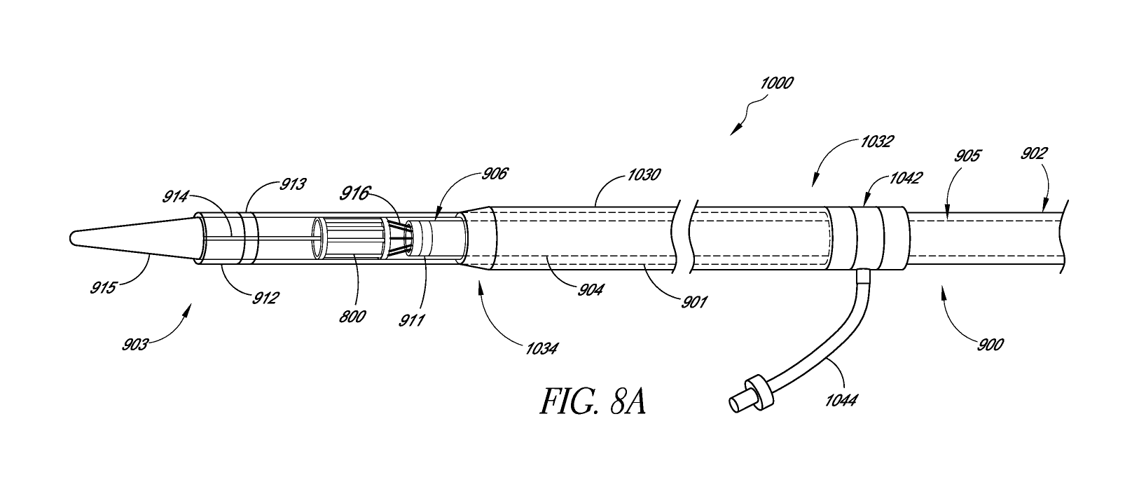

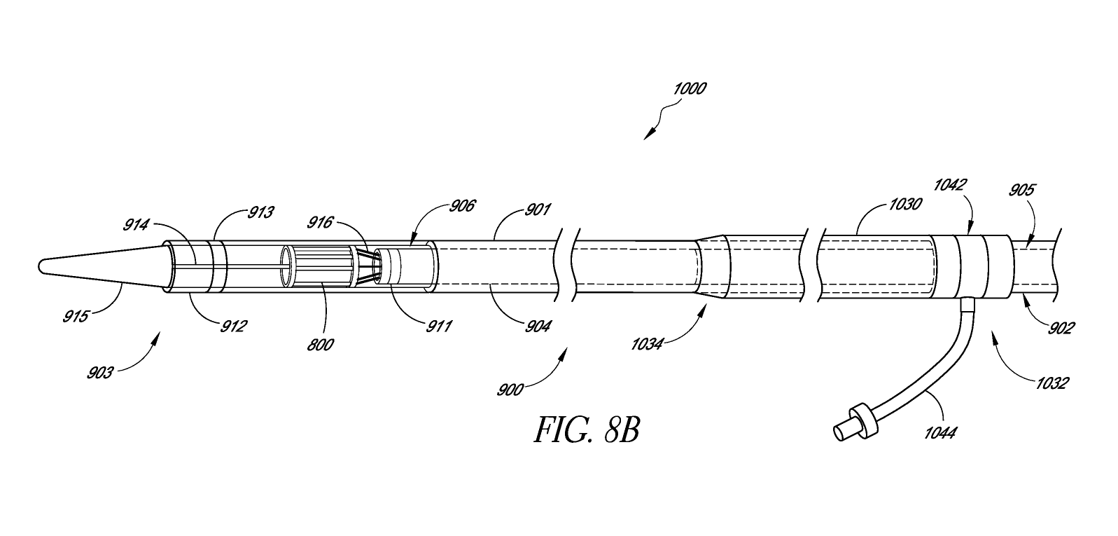

With reference to FIG. 3B, the inflation channels can be configured so that the cross-sectional profile of the implant 800 is reduced when it is compressed or in the retracted state. For example, the inflation channels 808 can be arranged in a step-function pattern. The inflation channels 808 can have three connection ports 809 for coupling to the delivery catheter 900 via position and fill lumen tubing (PFL) tubing 916 (see FIGS. 5A-5C). In some embodiments, at least two of the connection ports 809 also function as inflation ports, and inflation media, air or liquid can be introduced into the inflation channel 808 through these ports. The PFL tubing 916 can be connected to the connection ports 809 via suitable connection mechanisms. In one embodiment, the connection between the PFL tubing 916 and the connection port 809 is a screw connection. In some embodiments, an inflation valve 810 is present in the connection port 809 and can stop the inflation media, air or liquid from escaping the inflation channels 808 after the PFL tubing is disconnected. In some embodiments, the distal toroid 807b and the proximal toroid 807a can be inflated independently. In some embodiments, the distal toroid 807b can be inflated separately from the struts 806 and the proximal toroid 807a. The separate inflation can be useful during the positioning of the implant at the implantation site. With reference to FIGS. 3C and 3D, the portion of struts 806 can run parallel to the toroids 807 and can be encapsulated within the folds 801 of the implant 800. This arrangement may also aid in reducing the cross-sectional profile when the implant is compressed or folded.

As mentioned above, the inflatable rings or toroids 807 and struts 806 can form the inflatable structure 813, which, in turn, defines the inflation channels 808. The inflation channels 808 can receive inflation media to generally inflate the inflatable structure 813. When inflated, the inflatable rings 807 and struts 806 can provide structural support to the inflatable implant 800 and/or help to secure the implant 800 thin the heart 10. Uninflated, the implant 800 is a generally thin, flexible shapeless assembly that is preferably incapable of support and is advantageously able to take a small, reduced profile form in which it can be percutaneously inserted into the body. As will be explained in more detail below, in modified embodiments, the inflatable structure 813 can comprise any of a variety of configurations of inflation channels 808 that can be formed from other inflatable members in addition to or in the alternative to the inflatable rings 807 and struts 806 shown in FIGS. 3A and 3B. In one embodiment, the valve has an expanded diameter that is greater than or equal to 22 millimeters and a maximum compressed diameter that is less than or equal to 6 millimeters (18F).

With particular reference to FIG. 3B, in the illustrated embodiment, the distal ring 807b and struts 806 can be joined such that the inflation channel 808 of the distal ring 807b is in fluid communication with the inflation channel 808 of some of the struts 806. The inflation channel 808 of the proximal ring 807a can also be in communication with the inflation channels 808 of the proximal ring 807a and a few of the struts 806. In this manner, the inflation channels of the (i) proximal ring 807a and a few struts 806 can be inflated independently from the (ii) distal ring 807b and some struts. In some embodiments, the inflation channel of the proximal ring 807a can be in communication with the inflation channel of the struts 806, while the inflation channel of the distal ring 807b is not in communication with the inflation channel of the struts. As will be explained in more detail below, the two groups of inflation channels 808 can be connected to independent PFL tubing 916 to facilitate the independent inflation. It should be appreciated that in modified embodiments the inflatable structure can include less (i.e., one common inflation channel) or more independent inflation channels. For example, in one embodiment, the inflation channels of the proximal ring 807a, struts 806 and distal ring 807b can all be in fluid communication with each other such that they can be inflated from a single inflation device. In another embodiment, the inflation channels of the proximal ring 807a, struts 806 and distal ring 807b can all be separated and therefore utilize three inflation devices.

With reference to FIG. 3B, in the illustrated embodiment, each of the proximal ring 807a and the distal ring 807b can have a cross-sectional diameter of about 0.090 inches. The struts can have a cross-sectional diameter of about 0.060 inches. In some embodiments, within the inflation channels 808 are also housed valve systems that allow for pressurization without leakage or passage of fluid in a single direction. In the illustrated embodiment shown in FIG. 3B, two end valves or inflation valves 810 can reside at each end section of the inflation channels 808 adjacent to the connection ports 809. These end valves 810 are utilized to fill and exchange fluids such as saline, contrast agent and inflation media. The length of this inflation channel 808 can vary depending upon the size of the implant 800 and the complexity of the geometry. The inflation channel material can be blown using heat and pressure from materials such as nylon, polyethylene, Pebax, polypropylene or other common materials that will maintain pressurization. The fluids that are introduced are used to create the support structure, where without them, the implant 800 can be an undefined fabric and tissue assembly. In one embodiment the inflation channels 808 are first filled with saline and contrast agent for radiopaque visualization under fluoroscopy. This can make positioning the implant 800 at the implantation site easier. This fluid is introduced from the proximal end of the catheter 900 with the aid of an inflation device such as an endoflator or other devices to pressurize fluid in a controlled manner. This fluid can be transferred from the proximal end of the catheter 900 through the PFL tubes 916 which are connected to the implant 800 at the end of each inflation channel 808 at the connection port 809.

With reference to FIG. 3B, in the illustrated embodiment, the inflation channel 808 can have an end valve 810 (i.e., inflation valve) at each end whereby they can be separated from the PFL tubes 916 thus disconnecting the catheter from the implant. This connection can be a screw or threaded connection, a colleting system, an interference fit or other devices and methods of reliable securement between the two components (i.e., the end valve 810 and the PFL tubes 916). In between the ends of the inflation channel 808 can be an additional directional valve 811 to allow fluid to pass in a single direction. This allows for the filling of each end of the inflation channel 808 and displacement of fluid in a single direction. Once the implant 808 is placed at the desired position while inflated with saline and contrast agent, this fluid can be displaced by an inflation media that can solidify or harden. As the inflation media can be introduced from the proximal end of the catheter 900, the fluid containing saline and contrast agent is pushed out from one end of the inflation channel 808. Once the inflation media completely displaces the first fluid, the PFL tubes can then be disconnected from the implant 800 while the implant 800 remains inflated and pressurized. The pressure can be maintained in the implant 800 by the integral valve (i.e., end valve 810) at each end of the inflation channel 808. In the illustrated embodiment, this end valve 810 can have a ball 303 and seat to allow for fluid to pass when connected and seal when disconnected. In some case the implant 800 has three or more connection ports 809, but only two have inflation valves 810 attached. The connection port without the end valve 810 can use the same attachment device such as a screw or threaded element. Since, the illustrated embodiment, this connection port is not used for communication with the support structure 813 and its filling, no inflation valve 810 is necessary. In other embodiments, all three connection ports 809 can have inflation valves 810 for introducing fluids or inflation media.

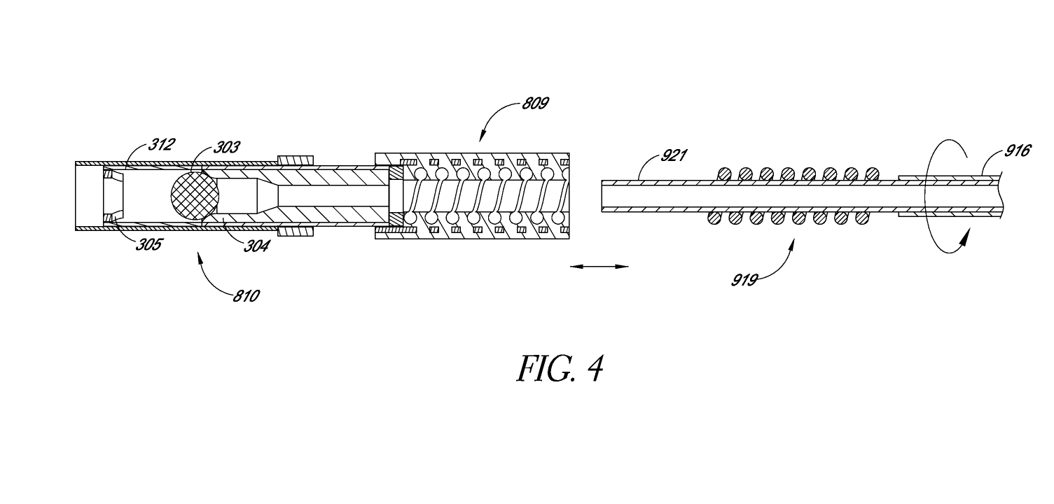

With reference to FIG. 4, the end valve system 810 can comprise a tubular section 312 with a soft seal 304 and spherical ball 303 to create a sealing mechanism 313. The tubular section 312 in one embodiment is about 0.5 cm to about 2 cm in length and has an outer diameter of about 0.010 inches to about 0.090 inches with a wall thickness of about 0.005 inches to about 0.040 inches. The material can include a host of polymers such as nylon, polyethylene, Pebax, polypropylene or other common materials such as stainless steel, Nitinol or other metallic materials used in medical devices. The soft seal material can be introduced as a liquid silicone or other material where a curing occurs thus allowing for a through hole to be constructed by coring or blanking a central lumen through the seal material. The soft seal 304 can be adhered to the inner diameter of the wall of the tubular member 312 with a through hole for fluid flow. The spherical ball 303 can move within the inner diameter of the tubular member 312 where it seats at one end sealing pressure within the inflation channels and is moved the other direction with the introduction of the PFL tube 916 but not allowed to migrate too far as a stop ring or ball stopper 305 retains the spherical ball 303 from moving into the inflation channel 808. As the PFL tube 916 is screwed into the connection port 809, the spherical ball 303 is moved into an open position to allow for fluid communication between the inflation channel 808 and the PFL tube 916. When disconnected, the ball 303 ca move against the soft seal 304 and halt any fluid communication external to the inflation channel 808 leaving the implant 800 pressurized. Additional embodiments can utilize a spring mechanism to return the ball to a sealed position and other shapes of sealing devices may be used rather than a spherical ball. A duck-bill style sealing mechanism or flap valve can also be used to halt fluid leakage and provide a closed system to the implant. Additional end valve systems have been described in U.S. Patent Publication No. 2009/0088836 to Bishop et al., which is thereby incorporated by reference herein.

The implant 800 of the illustrated embodiment ca allow delivery a prosthetic valve via catheterization in a lower profile and a safer manner than currently available. When the implant 800 is delivered to the site via a delivery catheter 900, the implant 800 is a thin, generally shapeless assembly in need of structure and definition. At the implantation site, the inflation media (e.g., a fluid or gas) can be added via PFL tubes of the delivery catheter 900 to the inflation channels 808 providing structure and definition to the implant 800. The inflation media therefore can comprise part of the support structure for implant 800 after it is inflated. The inflation media that is inserted into the inflation channels 808 can be pressurized and/or can solidify in situ to provide structure to the implant 800. Additional details and embodiments of the implant 800, can be found in U.S. Pat. No. 5,554,185 to Block and U.S. Patent Publication No. 2006/0088836 to Bishop et al., the disclosures of which are expressly incorporated by reference in their entirety herein.

The cuff 802 can be made from many different materials such as Dacron, TFE, PTFE, ePTFE, woven metal fabrics, braided structures, or other generally accepted implantable materials. These materials may also be cast, extruded, or seamed together using heat, direct or indirect, sintering techniques, laser energy sources, ultrasound techniques, molding or thermoforming technologies. Since the inflation channels 808 generally surrounds the cuff 802, and the inflation channels 808 can be formed by separate members (e.g., balloons and struts), the attachment or encapsulation of these inflation channels 808 can be in intimate contact with the cuff material. In some embodiments, the inflation channels 808 are encapsulated in the folds 801 or lumens made from the cuff material sewn to the cuff 802. These inflation channels 808 can also be formed by sealing the cuff material to create an integral lumen from the cuff 802 itself. For example, by adding a material such as a silicone layer to a porous material such as Dacron, the fabric can resist fluid penetration or hold pressures if sealed. Materials can also be added to the sheet or cylinder material to create a fluid-tight barrier.

Various shapes of the cuff 802 can be manufactured to best fit anatomical variations from person to person. As described above, these may include a simple cylinder, a hyperboloid, a device with a larger diameter in its mid portion and a smaller diameter at one or both ends, a funnel type configuration or other conforming shape to native anatomies. The shape of the implant 800 is preferably contoured to engage a feature of the native anatomy in such a way as to prevent the migration of the device in a proximal or distal direction. In one embodiment the feature that the device engages is the aortic root or aortic bulb 34 (see e.g., FIG. 2A), or the sinuses of the coronary arteries. In another embodiment the feature that the device engages is the native valve annulus, the native valve or a portion of the native valve. In certain embodiments, the feature that the implant 800 engages to prevent migration has a diameter difference between 1% and 10%. In another embodiment, the feature that the implant 800 engages to prevent migration the diameter difference is between 5% and 40%. In certain embodiments the diameter difference is defined by the free shape of the implant 800. In another embodiment the diameter difference prevents migration in only one direction. In another embodiment, the diameter difference prevents migration in two directions, for example proximal and distal or retrograde and antigrade. Similar to surgical valves, the implant 800 will vary in diameter ranging from about 14 mm to about 30 mm and have a height ranging from about 10 mm to about 30 mm in the portion of the implant 800 where the leaflets of the valve 104 are mounted. Portions of the implant 800 intended for placement in the aortic root can have larger diameters preferably ranging from about 20 mm to about 45 mm. In some embodiment, the implant 800 can have an outside diameter greater than about 22 mm when fully inflated.

In certain embodiments, the cuffs, inflated structure can conform (at least partially) to the anatomy of the patient as the implant 800 is inflated. Such an arrangement may provide a better seal between the patient's anatomy and the implant 800.

Different diameters of prosthetic valves may be needed to replace native valves of various sizes. For different locations in the anatomy, different lengths of prosthetic valves or anchoring devices will also be required. For example a valve designed to replace the native aortic valve needs to have a relatively short length because of the location of the coronary artery ostium (left and right arteries). A valve designed to replace or supplement a pulmonary valve could have significantly greater length because the anatomy of the pulmonary artery allows for additional length. Different anchoring mechanisms that may be useful for anchoring the implant 800 have been described in U.S. Patent Publication No. 2009/0088836 to Bishop et al.

In the embodiments described herein, the inflation channels 808 can be configured such that they are of round, oval, square, rectangular or parabolic shape in cross section. Round cross sections may vary from about 0.020-about 0.100 inches in diameter with wall thicknesses ranging from about 0.0005-about 0.010 inches. Oval cross sections may have an aspect ratio of two or three to one depending upon the desired cuff thickness and strength desired. In embodiments in which the inflation channels 808 are formed by balloons, these channels 808 can be constructed from conventional balloon materials such as nylon, polyethylene, PEEK, silicone or other generally accepted medical device material

In some embodiments, portions of the cuff or body 802 can be radio-opaque to aid in visualizing the position and orientation of the implant 800. Markers made from platinum gold or tantalum or other appropriate materials may be used. These may be used to identify critical areas of the valve that must be positioned appropriately, for example the valve commissures may need to be positioned appropriately relative to the coronary arteries for an aortic valve. Additionally during the procedure it may be advantageous to catheterize the coronary arteries using radio-opaque tipped guide catheters so that the ostium can be visualized. Special catheters could be developed with increased radio-opacity or larger than standard perfusion holes. The catheters could also have a reduced diameter in their proximal section allowing them to be introduced with the valve deployment catheter.

As mentioned above, during delivery, the body 802 can be limp and flexible providing a compact shape to fit inside a delivery sheath. The body 802 is therefore preferably made form a thin, flexible material that is biocompatible and may aid in tissue growth at the interface with the native tissue. A few examples of material may be Dacron, ePTFE, PTFE, TFE, woven material such as stainless steel, platinum, MP35N, polyester or other implantable metal or polymer. As mentioned above with reference to FIG. 2A, the body 802 may have a tubular or hyperboloid shape to allow for the native valve to be excluded beneath the wall of the cuff 802. Within this cuff 802 the inflation channels 808 can be connected to a catheter lumen for the delivery of an inflation media to define and add structure to the implant 800. The valve 104, which is configured such that a fluid, such as blood, may be allowed to flow in a single direction or limit flow in one or both directions, is positioned within the cuff 802. The attachment method of the valve 104 to the cuff 802 can be by conventional sewing, gluing, welding, interference or other devices and methods generally accepted by industry.

In one embodiment, the cuff 802 would have a diameter of between about 15 mm and about 30 mm and a length of between about 6 mm and about 70 mm. The wall thickness would have an ideal range from about 0.01 mm to about 2 mm. As described above, the cuff 802 may gain longitudinal support in situ from members formed by inflation channels or formed by polymer or solid structural elements providing axial separation. The inner diameter of the cuff 802 may have a fixed dimension providing a constant size for valve attachment and a predictable valve open and closure function. Portions of the outer surface of the cuff 802 may optionally be compliant and allow the implant 800 to achieve interference fit with the native anatomy.

The implant 800 can have various overall shapes (e.g., an hourglass shape to hold the device in position around the valve annulus, or the device may have a different shape to hold the device in position in another portion of the native anatomy, such as the aortic root). Regardless of the overall shape of the implant 800, the inflatable channels 808 can be located near the proximal and distal ends 803, 804 of the implant 800, preferably forming a configuration that approximates a ring or toroid 807. These channels may be connected by intermediate channels designed to serve any combination of three functions: (i) provide support to the tissue excluded by the implant 800, (ii) provide axial and radial strength and stiffness to the 800, and/or (iii) to provide support for the valve 104. The specific design characteristics or orientation of the inflatable structure 813 can be optimized to better serve each function. For example if an inflatable channel 808 were designed to add axial strength to the relevant section of the device, the channels 808 would ideally be oriented in a substantially axial direction.

The cuff 802 and inflation channels 808 of the implant 800 can be manufactured in a variety of ways. In one embodiment the cuff 802 is manufactured from a fabric, similar to those fabrics typically used in endovascular grafts or for the cuffs of surgically implanted prosthetic heart valves. The fabric is preferably woven into a tubular shape for some portions of the cuff 802. The fabric may also be woven into sheets. In one embodiment, the yarn used to manufacture the fabric is preferably a twisted yarn, but monofilament or braided yarns may also be used. The useful range of yarn diameters is from approximately 0.0005 of an inch in diameter to approximately 0.005 of an inch in diameter. Depending on how tight the weave is made. Preferably, the fabric is woven with between about 50 and about 500 yarns per inch. In one embodiment, a fabric tube is woven with a 18 mm diameter with 200 yarns per inch or picks per inch. Each yarn is made of 20 filaments of a PET material. The final thickness of this woven fabric tube is 0.005 inches for the single wall of the tube. Depending on the desired profile of the implant 800 and the desired permeability of the fabric to blood or other fluids different weaves may be used. Any biocompatible material may be used to make the yarn, some embodiments include nylon and PET. Other materials or other combinations of materials are possible, including Teflon, fluoropolymers, polyimide, metals such as stainless steel, titanium, Nitinol, other shape memory alloys, alloys comprised primarily of a combinations of cobalt, chromium, nickel, and molybdenum. Fibers may be added to the yarn to increases strength or radiopacity, or to deliver a pharmaceutical agent. The fabric tube may also be manufactured by a braiding process.

The fabric can be stitched, sutured, sealed, melted, glued or bonded together to form the desired shape of the implant 800. The preferred method for attaching portions of the fabric together is stitching. The preferred embodiment uses a polypropylene monofilament suture material, with a diameter of approximately 0.005 of an inch. The suture material may range from about 0.001 to about 0.010 inches in diameter. Larger suture materials may be used at higher stress locations such as where the valve commissures attach to the cuff. The suture material may be of any acceptable implant grade material. Preferably a biocompatible suture material is used such as polypropylene. Nylon and polyethylene are also commonly used suture materials. Other materials or other combinations of materials are possible, including Teflon, fluoropolymers, polyimides, metals such as stainless steel, titanium, Kevlar, Nitinol, other shape memory alloys, alloys comprised primarily of a combinations of cobalt, chromium, nickel, and molybdenum such as MP35N. Preferably the sutures are a monofilament design. Multi strand braided or twisted suture materials also may be used. Many suture and stitching patterns are possible and have been described in various texts. The preferred stitching method is using some type of lock stitch, of a design such that if the suture breaks in a portion of its length the entire running length of the suture will resist unraveling. And the suture will still generally perform its function of holding the layers of fabric together.

In some embodiments, the implant 800 is not provided with separate balloons, instead the fabric of the cuff 802 itself can form the inflation channels 808. For example, in one embodiment two fabric tubes of a diameter similar to the desired final diameter of the implant 800 are place coaxial to each other. The two fabric tubes are stitched, fused, glued or otherwise coupled together in a pattern of channels 808 that is suitable for creating the geometry of the inflatable structure 813. In some embodiments, the fabric tubes are sewn together in a pattern so that the proximal and distal ends of the fabric tubes form an annular ring or toroid 807. In some embodiments, the middle section of the implant 800 contains one or more inflation channels shaped in a step-function pattern. In some embodiments, the fabric tubes are sewn together at the middle section of the implant to form inflation channels 808 that are perpendicular to the toroids 807 at the end sections of the implant 800. Methods for fabricating the implant 800 have been described in U.S. Patent Publication No. 2006/0088836 to Bishop et al.

In the illustrated embodiment of FIGS. 3A and 3B, the struts 806 are arranged such that there is no radial overlap with the distal and proximal rings 807a, 807b. That is, in the illustrated embodiment, the struts 808 do not increase the radial thickness of the inflation structure because there is no radial overlap between the distal and proximal rings and the channels so that the channels lie within the radial thickness envelop defined by the distal and proximal rings 807a, 807b. In another embodiment, the struts 808 can be wider in the radial direction than the distal and proximal rings 807a, 807b such that the distal and proximal rings 807a, 807b lie within a radial thickness envelop defined by the struts 806.

In one embodiment, the valve 800 can be delivered through a deployment catheter with an 18 F or smaller outer diameter and when fully inflated has an effective orifice area of at least about 1.0 square cm; and in another embodiment at least about 1.3 square cm and in another embodiment about 1.5 square cm. In one embodiment, the valve 800 has a minimum cross-sectional flow area of at least about 1.75 square cm.

### Leaflet Subassembly

With reference back to the embodiments of FIG. 2A, the valve 104 preferably is a tissue-type heart valve that includes a dimensionally stable, pre-aligned tissue leaflet subassembly. Pursuant to this construction, an exemplary tissue valve 104 can include a plurality of tissue leaflets that are templated and attached together at their tips to form a dimensionally stable and dimensionally consistent coapting leaflet subassembly. Then, in what can be a single process, each of the leaflets of the subassembly can be aligned with and individually sewn to the cuff 802, from the tip of one commissure uniformly, around the leaflet cusp perimeter, to the tip of an adjacent commissure. As a result, the sewed sutures act like similarly aligned staples, all of which equally take the loading force acting along the entire cusp of each of the pre-aligned, coapting leaflets. Once inflated, the cuff 802 can support the commissures with the inflation media and its respective pressure which will solidify and create a system similar to a stent structure. The resulting implant 800 thereby formed can reduce stress and potential fatigue at the leaflet suture interface by distributing stress evenly over the entire leaflet cusp from commissure to commissure. In some embodiments, the tissue valve is coupled to the inflatable cuff 802 by attaching to the fabric of the cuff only.

In one embodiment, the tissue leaflets are not coupled to each other but are instead individually attached to the cuff 802.

A number of additional advantages can result from the use of the implant 800 and the cuff 802 construction utilized therein. For example, for each key area of the cuff 802, the flexibility can be optimized or customized. If desired, the coapting tissue leaflet commissures can be made more or less flexible to allow for more or less deflection to relieve stresses on the tissue at closing or to fine tune the operation of the valve. Similarly, the base radial stiffness of the overall implant structure can be increased or decreased by pressure or inflation media to preserve the roundness and shape of the implant 800.

Attachment of the valve 104 to the cuff 802 can be completed in any number of conventional methods including sewing, ring or sleeve attachments, gluing, welding, interference fits, bonding through mechanical devices and methods such as pinching between members. An example of these methods are described in Published Applications from Huynh et al (06/102944) or Lafrance et al (2003/0027332) or U.S. Pat. No. 6,409,759 to Peredo, which are hereby incorporated by reference herein. These methods are generally know and accepted in the valve device industry. The valve, whether it is tissue, engineered tissue, mechanical or polymer, may be attached before packaging or in the hospital just before implantation. Some tissue valves are native valves such as pig, horse, cow or native human valves. Most of which are suspended in a fixing solution such as Glutaraldehyde.

In some embodiments, heart valve prostheses can be constructed with flexible tissue leaflets or polymer leaflets. Prosthetic tissue heart valves can be derived from, for example, porcine heart valves or manufactured from other biological material, such as bovine or equine pericardium. Biological materials in prosthetic heart valves generally have profile and surface characteristics that provide laminar, nonturbulent blood flow. Therefore, intravascular clotting is less likely to occur than with mechanical heart valve prostheses.

Natural tissue valves can be derived from an animal species, typically mammalian, such as human, bovine, porcine canine, seal or kangaroo. These tissues can be obtained from, for example, heart valves, aortic roots, aortic walls, aortic leaflets, pericardial tissue such as pericardial patches, bypass grafts, blood vessels, human umbilical tissue and the like. These natural tissues are typically soft tissues, and generally include collagen containing material. The tissue can be living tissue, decellularized tissue or recellularized tissue. Tissue can be fixed by crosslinking. Fixation provides mechanical stabilization, for example by preventing enzymatic degradation of the tissue. Glutaraldehyde or formaldehyde is typically used for fixation, but other fixatives can be used, such as other difunctional aldehydes, epoxides, genipin and derivatives thereof. Tissue can be used in either crosslinked or uncrosslinked form, depending on the type of tissue, use and other factors. Generally, if xenograft tissue is used, the tissue is crosslinked and/or decellularized. Additional description of tissue valves can be found in U.S. Patent Publication No. 2009/008836 to Bishop et al.

### Inflation Media

The inflatable structure 813 can be inflated using any of a variety of inflation media, depending upon the desired performance. In general, the inflation media can include a liquid such water or an aqueous based solution, a gas such as CO₂, or a hardenable media which may be introduced into the inflation channels 808 at a first, relatively low viscosity and converted to a second, relatively high viscosity. Viscosity enhancement may be accomplished through any of a variety of known UV initiated or catalyst initiated polymerization reactions, or other chemical systems known in the art. The end point of the viscosity enhancing process may result in a hardness anywhere from a gel to a rigid structure, depending upon the desired performance and durability.

Useful inflation media generally include those formed by the mixing of multiple components and that have a cure time ranging from a tens of minutes to about one hour, preferably from about twenty minutes to about one hour. Such a material may be biocompatible, exhibit long-term stability (preferably on the order of at least ten years in vivo), pose as little an embolic risk as possible, and exhibit adequate mechanical properties, both pre and post-cure, suitable for service in the cuff in vivo. For instance, such a material should have a relatively low viscosity before solidification or curing to facilitate the cuff and channel fill process. A desirable post-cure elastic modulus of such an inflation medium is from about 50 to about 400 psi—balancing the need for the filled body to form an adequate seal in vivo while maintaining clinically relevant kink resistance of the cuff. The inflation media ideally should be radiopaque, both acute and chronic, although this is not absolutely necessary.

One preferred family of hardenable inflation media are two part epoxies. The first part is an epoxy resin blend comprising a first aromatic diepoxy compound and a second aliphatic diepoxy compound. The first aromatic diepoxy compound provides good mechanical and chemical stability in an aqueous environment while being soluble in aqueous solution when combined with suitable aliphatic epoxies. In some embodiments, the first aromatic diepoxy compound comprises at least one N,N-diglycidylaniline group or segment. In some embodiments, the first aromatic diepoxy compound are optionally substituted N,N-diglycidylaniline. The substitutent may be glycidyloxy or N,N-diglycidylanilinyl-methyl. Non-limiting examples of the first aromatic diepoxy compound are N,N-diglycidylaniline, N,N-diclycidyl-4-glycidyloxyaniline (DGO) and 4,4′-methylene-bis(N,N-diglycidylaniline) (MBD), etc.

The second aliphatic diepoxy compound provides low viscosity and good solubility in an aqueous solution. In some embodiments, the second aliphatic diepoxy compound may be 1,3-butadiene diepoxide, glycidyl ether or C₁₋₅ alkane diols of glycidyl ether. Non-limiting examples of the second aliphatic diepoxy compounds are 1,3-butadiene diepoxide, butanediol diglycidyl ether (BDGE), 1,2-ethanediol diglycidyl ether, glycidyl ether, etc.

In some embodiments, additional third compound may be added to the first part epoxy resin blend for improving mechanical properties and chemical resistance. In some embodiments, the additional third compound may be an aromatic epoxy other than the one containing N,N-diglycidylanaline. However, the solubility of the epoxy resin blend can also decrease and the viscosity can increase as the concentration of the additional aromatic epoxies increases. The preferred third compound may be tris(4-hydroxyphenyl)methane triglycidyl ether (THTGE), bisphenol A diglycidyl ether (BADGE), bisphenol F diglycidyl ether (BFDGE), or resorcinol diglycidyl ether (RDGE).

In some embodiments, the additional third compound may be a cycloaliphatic epoxy compound, preferably more soluble than the first aromatic diepoxy compound. It can increase the mechanical properties and chemical resistance to a lesser extent than the aromatic epoxy described above, but it will not decrease the solubility as much. Non-limiting examples of such cycloaliphatic epoxy are 1,4-cyclohexanedimethanol diclycidyl ether and cyclohexene oxide diglycidyl 1,2-cyclohexanedicarboxylate. Similarly, in some embodiments, aliphatic epoxy with 3 or more glycidyl ether groups, such as polyglycidyl ether, may be added as the additional third compound for the same reason. Polyglycidyl ether may increase cross linking and thus enhance the mechanical properties.

In general, the solubility of the epoxy resin blend decreases and the viscosity increases as the concentration of the first aromatic diepoxy compound increases. In addition, the mechanical properties and chemical resistance may be reduced as the concentration of the aliphatic diepoxy compound goes up in the epoxy resin blend. By adjusting the ratio of the first aromatic dipoxy compound and the second aliphatic diepoxy compound, a person skilled in the art can control the desired properties of the epoxy resin blend and the hardened media. Adding the third compound in some embodiments may allow further tailoring of the epoxy resin properties.

The second part of the hardenable inflation media comprises a hardener comprising at least one cycloaliphatic amine. It provides good combination of reactivity, mechanical properties and chemical resistance. The cycloaliphatic amine may include, but not limited to, isophorone diamine (IPDA), 1,3-bisaminocyclohexame (1,3-BAC), diamino cyclohexane (DACH), n-aminoethylpiperazine (AEP) or n-aminopropylpiperazine (APP).

In some embodiments, an aliphatic amine may be added into the second part to increase reaction rate, but may decrease mechanical properties and chemical resistance. The preferred aliphatic amine has the structural formula (I):

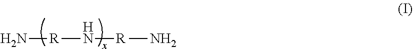

wherein each R is independently selected from branched or linear chains of C₂₋₅ alkyl, preferably C₂ alkyl. The term “alkyl” as used herein refers to a radical of a fully saturated hydrocarbon, including, but not limited to, methyl, ethyl, n-propyl, isopropyl (or i-propyl), n-butyl, isobutyl, tert-butyl (or t-butyl), n-hexyl, and the like. For example, the term “alkyl” as used herein includes radicals of fully saturated hydrocarbons defined by the following general formula CₙH₂ₙ₊₂. In some embodiments, the aliphatic amine may include, but not limited to, tetraehtylenepentamine (TEPA), diethylene triamine and triethylene tetraamine. In some embodiments, the hardener may further comprise at least one radio-opaque compound, such as iodo benzoic acids.

Additional details of hardenable inflation media are described in co-pending application titled “Inflation Media Formulation” application Ser. No. 13/110,780, filed May 18, 2011, the entirety of which is hereby incorporated herein by reference. Other suitable inflation media are also described in U.S. patent application Ser. No. 09/496,231 to Hubbell et al., filed Feb. 1, 2000, entitled “Biomaterials Formed by Nucleophilic Addition Reaction to Conjugated Unsaturated Groups” and U.S. Pat. No. 6,958,212 to Hubbell et al. The entireties of each of these patents are hereby incorporated herein by reference.

Below is listed one particular two-component medium. This medium comprises:

### First Part—Epoxy Resin Blend

(1) N,N-Diglycidyl-4-glycidyloxyaniline (DGO), present in a proportion ranging from about 10 to about 70 weight percent; specifically in a proportion of about 50 weight percent,

(2) Butanediol diglycidyl ether (BDGE) present in a proportion ranging from about 30 to about 75 weight percent; specifically in a proportion of about 50 weight percent, and optionally

(3) 1,4-Cyclohexanedimethanol diglycidyl ether, present in a proportion ranging from about 0 to about 50 weight percent.

### Second Part—Amine Hardener

(1) Isophorone diamine (IPDA), present in a proportion ranging from about 75 to about 100 weight percent, and optionally

(2) Diethylene triamine (DETA), present in a proportion ranging from about 0 to about 25 weight percent.

The mixed uncured inflation media preferably has a viscosity less than 2000 cps. In one embodiment the epoxy based inflation media has a viscosity of 100-200 cps. In another embodiment the inflation media has a viscosity less than 1000 cps. In some embodiments, the epoxy mixture has an initial viscosity of less than about 50 cps, or less than about 30 cps after mixing. In some embodiments, the average viscosity during the first 10 minutes following mixing the two components of the inflation media is about 50 cps to about 60 cps. The low viscosity ensures that the inflation media can be delivered through the inflation lumen of a deployment catheter with small diameter, such as an 18 French catheter

In some embodiments, the balloon or inflation channel may be connected to the catheter on both ends. This allows the balloon to be pre-inflated with a non-solidifying material such as a gas or liquid. If a gas is chosen, CO₂ or helium are the likely choices; these gasses are used to inflate intra-aortic balloon pumps. Preferably the pre-inflation media is radio-opaque so that the balloon position can be determined by angiography. Contrast media typically used in interventional cardiology could be used to add sufficient radio-opacity to most liquid pre-inflation media. When it is desired to make the implant permanent and exchange the pre-inflation media for the permanent inflation media, the permanent inflation media is injected into the inflation channel through a first catheter connection. In some embodiments, the permanent inflation media is capable of solidifying into a semi-solid, gel or solid state. As the permanent inflation media is introduced into the inflatable structure, the pre-inflation media is expelled out from a second catheter connection. The catheter connections are positioned in such a way that substantially all of the pre-inflation media is expelled as the permanent inflation media is introduced. In one embodiment an intermediate inflation media is used to prevent entrapment of pre-inflation media in the permanent inflation media. In one embodiment the intermediate inflation media is a gas and the pre-inflation media is a liquid. In another embodiment the intermediate inflation media or pre-inflation media functions as a primer to aid the permanent inflation media to bond to the inner surface of the inflation channel. In another embodiment the pre-inflation media or the intermediate inflation media serves as a release agent to prevent the permanent inflation media from bonding to the inner surface of the inflation channel.

The permanent inflation media may have a different radiopacity than the pre-inflation media. A device that is excessively radiopaque tends to obscure other nearby features under angiography. During the pre-inflation step it may be desirable to visualize the inflation channel clearly, so a very radiopaque inflation media may be chosen. After the device is inflated with the permanent inflation media a less radiopaque inflation media may be preferred. The feature of lesser radiopacity is beneficial for visualization of proper valve function as contrast media is injected into the ventricle or the aorta.

### Low Crossing Profile Delivery System

FIGS. 5A-5B illustrate an embodiment of a low crossing profile delivery catheter 900 that can be used to deliver the implant 800. In general, the delivery system comprises a delivery catheter 900, and the delivery catheter 900 can comprise an elongate, flexible catheter body having a proximal end and a distal end. In some embodiments, the catheter body has a maximum outer diameter of about 18 French or less particularly at the distal portion of the catheter body (i.e. the deployment portion). In some embodiments, the delivery catheter also comprises a cardiovascular prosthetic implant 800 (e.g., configured as described above) at the distal end of the catheter body. While using a cardiovascular prosthetic implant 800 as described above has certain advantages, in modified embodiments, certain features of the delivery catheter and delivery system described herein can also be used with a prosthetic implant that utilizes a stent or other support structure and/or does not utilize an inflation media.

As described herein, certain features of the implant 800 and delivery catheter 900 are particularly advantageous for facilitating delivering of cardiovascular prosthetic implant 800 within a catheter body having outer diameter of about 18 French or less while still maintaining a tissue valve thickness equal to or greater than about 0.011 inches and/or having an effective orifice area equal to or greater than about 1 cm squared, or in another embodiment, 1.3 cm squared or in another embodiment 1.5 cm squared. In such embodiments, the implant 800 can also have an expanded maximum diameter that is greater than or equal to about 22 mm. In some embodiments, at least one link exists between the catheter body and the implant 800. In some embodiments, the at least one link is the PFL tubing. In one embodiment, the delivery system is compatible with 0.035″ or 0.038″ guidewire.

In general, the delivery catheter 900 can be constructed with extruded tubing using well known techniques in the industry. In some embodiments, the catheter 900 can incorporates braided or coiled wires and or ribbons into the tubing for providing stiffness and rotational torqueability. Stiffening wires may number between 1 and 64. In some embodiments, a braided configuration is used that comprises between 8 and 32 wires or ribbon. If wires are used in other embodiments, the diameter can range from about 0.0005 inches to about 0.0070 inches. If a ribbon is used, the thickness is preferably less than the width, and ribbon thicknesses may range from about 0.0005 inches to about 0.0070 inches while the widths may range from about 0.0010 inches to about 0.0100 inches. In another embodiment, a coil is used as a stiffening member. The coil can comprise between 1 and 8 wires or ribbons that are wrapped around the circumference of the tube and embedded into the tube. The wires may be wound so that they are parallel to one another and in the curved plane of the surface of the tube, or multiple wires may be wrapped in opposing directions in separate layers. The dimensions of the wires or ribbons used for a coil can be similar to the dimensions used for a braid.

With reference to FIGS. 5A and 5B, the catheter 900 can comprise an outer tubular member 901 having a proximal end 902 and a distal end 903, and an inner tubular member 904 also having a proximal end 905 and a distal end 906. The inner tubular member 904 can extend generally through the outer tubular member 901, such that the proximal and distal ends 902, 903 of the inner tubular member 904 extend generally past the proximal end 902 and distal end 903 of the outer tubular member 901. The distal end 903 of the outer tubular member 901 can comprise a sheath jacket 912 and a stem region 917 that extends proximally from the sheath jacket 912. In some embodiments, the sheath jacket 912 may comprise KYNAR tubing. The sheath jacket 912 can house the implant 800 in a retracted state for delivery to the implantation site. In some embodiments, the sheath jacket 912 is capable of transmitting at least a portion of light in the visible spectrum. This allows the orientation of the implant 800 to be visualized within the catheter 900. In some embodiments, an outer sheath marking band 913 may be located at the distal end 903 of the outer tubular member 901.

In one embodiment, the sheath jacket 912 can have a larger outside diameter than the adjacent or proximate region of the stem region 917 of the tubular member 901. In such embodiments, the sheath jacket 917 and the stem region 917 can comprise separate tubular components that are attached or otherwise coupled to each other. In other embodiments, the tubular member 901 can be expanded to form the larger diameter sheath jacket 912 such that the stem region 917 and sheath jacket 912 are formed from a common tubular member. In another embodiment or in combination with the previous embodiments, the diameter of the stem region 917 can be reduced.

The proximal end 905 of the inner tubular member 904 can be connected to a handle 907 for grasping and moving the inner tubular member 904 with respect to the outer tubular member 901. The proximal end 902 of the outer tubular member 901 can be connected to an outer sheath handle 908 for grasping and holding the outer tubular member 901 stationary with respect to the inner tubular member 904. A hemostasis seal 909 can be preferably provided between the inner and outer tubular members 901, 904, and the hemostasis seal 909 can be disposed in outer sheath handle 908. In some embodiments, the outer sheath handle 908 comprises a side port valve 921, and the fluid can be passed into the outer tubular member through it.

In general, the inner tubular member 904 comprises a multi-lumen hypotube (see FIG. 6). In some embodiments, a neck section 910 is located at the proximal end 905 of the inner tubular member 904. The neck section 910 may be made from stainless steel, Nitinol or another suitable material which can serve to provide additional strength for moving the inner tubular member 904 within the outer tubular member 901. In some embodiments, a marker band 911 is present at the distal end 906 of the inner tubular member 904. The multi-lumen hypotube can have a wall thickness between about 0.004 in and about 0.006 in. In one embodiment, the wall thickness is about 0.0055 in, which provides sufficient column strength and increases the bending load required to kink the hypotube. With reference to FIG. 6, the inner tubular member 904 (i.e., multi-lumen hypotube in the illustrated embodiment) can comprise at least four lumens. One of the lumens can accommodate the guidewire tubing 914, and each of the other lumens can accommodate a positioning-and-fill lumen (PFL) tubing 916. The guidewire tubing 914 can be configured to receive a guidewire. The PFL tubing 916 can be configured to function both as a control wire for positioning the implant 800 at the implantation cite, and as an inflation tube for delivering a liquid, gas or inflation media to the implant 800. In particular, the tubing 916 can allow angular adjustment of the implant 800. That is, the plane of the valve (defined generally perpendicular to the longitudinal axis of the implant 800) can be adjusted with the tubing 916.

With reference to FIGS. 5A and 5B, in general, the guidewire tubing 914 can be longer than and can extend throughout the length of the delivery catheter 900. The proximal end of the guidewire tubing 914 can pass through the inner sheath handle 907 for operator's control; the distal end of the guidewire tubing 914 can extend past the distal end 903 of the outer tubular member 901, and can be coupled to a guidewire tip 915. The guidewire tip 915 can close the distal end 903 of the outer tubular member 901 (or the receptacle) and protect the retracted implant 800, for example, during the advancement of the delivery catheter. The guidewire tip 915 can be distanced from the outer tubular member 901 by proximally retracting the outer tubular member 901 while holding the guidewire tubing 914 stationary. Alternatively, the guidewire tubing 914 can be advanced while holding the outer tubular member 901 stationary. The guidewire tubing 914 can have an inner diameter of about 0.035 inches to about 0.042 inches, so the catheter system is compatible with common 0.035″ or 0.038″ guidewires. In some embodiments, the guidewire tubing 914 may have an inner diameter of about 0.014 inches to about 0.017 inches, so the catheter system is compatible with a 0.014″ diameter guidewire. The guidewire tubing 914 can be made from a lubricious material such as Teflon, polypropylene or a polymer impregnated with Teflon. It can also be coated with a lubricous or hydrophilic coating.

The guidewire tip 915 may be cone shaped, bullet shaped or hemispherical on the front end. The largest diameter of the guidewire tip 915 is preferably approximately the same as the distal portion 903 of the outer tubular member 901. The guidewire tip 915 preferably steps down to a diameter slightly smaller than the inside diameter of the outer sheath jacket 912, so that the tip can engage the outer sheath jacket 912 and provide a smooth transition. In the illustrated embodiment, the guidewire tip 915 is connected to the guidewire tube 914, and the guidewire lumen passes through a portion of the guidewire tip 915. The proximal side of the guidewire tip 915 also has a cone, bullet or hemispherical shape, so that the guidewire tip 915 can easily be retraced back across the deployed implant 800, and into the deployment catheter 900. The guidewire tip 915 can be manufactured from a rigid polymer such as polycarbonate, or from a lower durometer material that allows flexibility, such as silicone. Alternatively, the guidewire tip 915 may be made from multiple materials with different durometers. For example, the portion of the guidewire tip 915 that engages the distal portion 903 of the outer tubular member 901 can be manufactured from a rigid material, while the distal and or proximal ends of the guidewire tip 915 are manufactured from a lower durometer material.

As will be explained in detail below, in one embodiment, the guidewire tip 915 is configured (e.g., has a tapered shape) to for direct insertion into an access vessel over a guidewire. In this manner, the guidewire tip 915 and the jacket 912 can be used to directly dilate the access vessel to accommodate an introducer catheter positioned over the delivery catheter.

Each PFL tubing 916 can extend throughout the length of the delivery catheter 900. The proximal end of the PFL tubing 916 passes through the handle 907, and has a luer lock 917 for connecting to fluid, gas or inflation media source. The distal end of the PFL tubing 916 extends past the distal end 906 of the inner tubular member 904 through the hypotube lumen. With reference to FIG. 5C, in some embodiments, the PFL tubing 916 comprises a strain relief section 918 at the proximal end where the tubing 916 is connected to the luer lock 917, and the strain relief section 918 serves to relieve the strain on the PFL tubing 916 while being maneuvered by the operator. The distal end of the PFL tubing 916 comprises a tip or needle 919 for connecting to the implant 800. In some embodiments, the tip 919 may have a threaded section toward the end of the needle 919 (see FIG. 5C). In some embodiments, the PFL tubing 916 may have PFL marker(s) 920 at the distal end and/or proximal end of the tubing 916 for identification.

The PFL tubing 916 can be designed to accommodate for the ease of rotation in a tortuous anatomy. The tubing 916 may be constructed using polyimide braided tube, Nitinol hypotube, or stainless steel hypotube. In a preferred embodiment, the PFL tubing 916 is made from braided polyimide, which is made of polyimide liner braided with flat wires, encapsulated by another polyimide layer and jacketed with prebax and nylon outer layer. In some embodiments, a Nitinol sleeve can be added to the proximal end of the PFL tubing 916 to improve torque transmission, kinks resistance and pushability. In some embodiments, the outside surface of the PFL tubing 916 and/or the inside surface of the lumens in the multi-lumen hypotube can also be coated with a lubricious silicone coating to reduce friction. In some embodiments, an inner lining material such as Teflon can be used on the inside surface of the lumens in the multi-lumen hypotube to reduce friction and improve performance in tortuous curves. Additionally, slippery coatings such as DOW 360, MDX silicone or a hydrophilic coating from BSI Corporation may be added to provide another form of friction reducing elements. This can provide a precision control of the PFL tubings 916 during positioning of the implant 800. In some embodiments, the outside surface of the PFL tubing 916 can be jacketed and reflowed with an additional nylon 12 or Relsan AESNO layer to ensure a smooth finished surface. In some embodiments, anti-thrombus coating can also be put on the outside surface of the PFL tubing 916 to reduce the risk of thrombus formation on the tubing. In some embodiments, the PFL tubing 916 can have a textured coating that can make the PFL tubing 916 easier to hold or manipulate. The textured coating can also be selected to increase the pushability of the wire.

In some embodiments, the outer diameter of the catheter 900 can measure between about 0.030 inches to about 0.200 inches with a wall thickness of the outer tubular member 901 being about 0.005 inches to about 0.060 inches. In certain embodiments, the outer diameter of the outer tubular member 901 can be between about 0.215 and about 0.219 inches. In this embodiment, the wall thickness of the outer tubular member 901 is between about 0.005 inches and about 0.030 inches. The overall length of the catheter 900 can range from about 80 centimeters to about 320 centimeters. In certain embodiments, the working length of the outer tubular member 901 (from the distal end of the sheath jacket 912 to the location where the tubular member 901 is connected to the outer sheath handle 908) can be about 100 cm to about 120 cm. In some embodiments, the inner diameter of the sheath jacket 912 can be greater than or equal to about 0.218 inches, and the outer diameter of the sheath jacket 912 is less than or equal to about 0.241 inches. In a preferred embodiment, the outer diameter of the sheath jacket portion 912 can be less than or equal to about 0.236 inches or 18 French. In some embodiments, the outer diameter of the PFL tubing 916 can be less than or equal to about 0.0435 inches, and the length is about 140 cm to about 160 cm. In some embodiments, at least a portion of the PFL tubing 916 can have an increased diameter, e.g. the transverse diameter of the PFL tubing 916 can be 0.050 inches.

In the embodiments that employ a low crossing profile outer tubular member, a low profile inflatable implant in a retracted state is preferable for fitting into the sheath jacket 912. The sheath jacket 912 can have an outer diameter of 18 French or less. In some embodiments, the implant 800 comprises a tissue valve 104 with an expanded outer diameter greater than or equal to about 22 mm and a tissue thickness of greater than or equal to about 0.011 inches. The compressed diameter of the implant 800 may be less than or equal to about 6 mm or 18 French. The retracted implant 800 is generally loaded between the distal portion 903 of the outer tubular member 901 and the distal portion 906 of the inner tubular member 904. The distal portion 903 of the outer tubular member 901 therefore can form a receptacle for the implant 800. The implant 800 can be exposed or pushed out of the receptacle by holding the implant 800 stationary as the outer tubular member 901 is retracted. Alternatively, the outer tubular member 901 can be held stationary while the inner tubular member 904 is advanced and thereby pushing the implant 800 out of the receptacle.

The delivery system can include a loading tool base 925 that can connect to the PFL tubing 916. In some embodiments, the PFL tubing 916 can connect to the loading tool base 921 via a luer connection. With reference to FIG. 7, one end of the loading tool base 921 can be configured to have step edge 923s. In some embodiments, the distal end of the loading tool base has three step edges 923, each step edge 923 has a luer connector 924 for connecting the PFL tubing 916. In some embodiments, the loading tool base 921 can also comprise at least two additional connectors 922 (e.g. additional luer connectors), each in fluid communication with one of the luer connector 924 on the stepped edges 923, which would allow the introduction of fluid, gas or air into the implant 800 for testing purposes. For example, in the exemplified embodiment, once the PFL tubings 916 are connected to the loading tool base 921, a liquid or air source can be connected to the loading tool base 921 via the additional connectors 922. The liquid or air can then be introduced into the implant 800 through the loading tool base 921 and the PFL tubings 916.

The step edges 923 on the loading tool base 921 can allow the implant 800 to be collapsed or folded up tightly so it can be loaded into the sheath jacket 912 at the distal end of the outer tubular member 901. When the proximal end of the PFL tubings 916 are connected to the loading tool base 921 and the distal end connected to the connection ports 809 of the implant 800, the step edge connections can pull the PFL tubings 916 in a way that creates an offset of the inflation valves 810 and/or the connection ports 809 in the inflation channels 808 when the implant 800 is folded or collapsed. By staggering the connection ports/inflation valves, the collapsed implant 800 can have a reduced cross-sectional profile. In some embodiments, the check valve 814 in the inflation channel is also staggered with the connection ports/inflation valves in the collapsed state. Accordingly, in one embodiment, the inflation valves 810 and/or the connection ports 809 are axially aligned when the valve is positioned within the deployment catheter in a collapsed configuration. That is, the inflation valves 810 and/or the connection ports 809 and/or check valve 814 are positioned such that they do not overlap with each other but are instead aligned generally with respect to the longitudinal axis of the deployment catheter. In this manner, the implant 800 can be collapsed into a smaller diameter as opposed to a configuration in which with the inflation valves 810 and/or the connection ports 809 and/or check valve 814 overlap each other in a radial direction, which can increase the diameter of the compressed implant 800. In a similar manner, the channels 806 can be arranged positioned such hat they also do not overlap with each other. The loading tool base 925 can be used to pull one end of the distal and proximal rings 807a, 807b in a proximal direction so as to align the inflation valves 810 and/or the connection ports 809 and/or check valve 814 axially as described above and/or align the channels so as to reduce the overlap between multiple channels 806.

Combined Delivery System with Delivery Catheter and Introducer Catheter

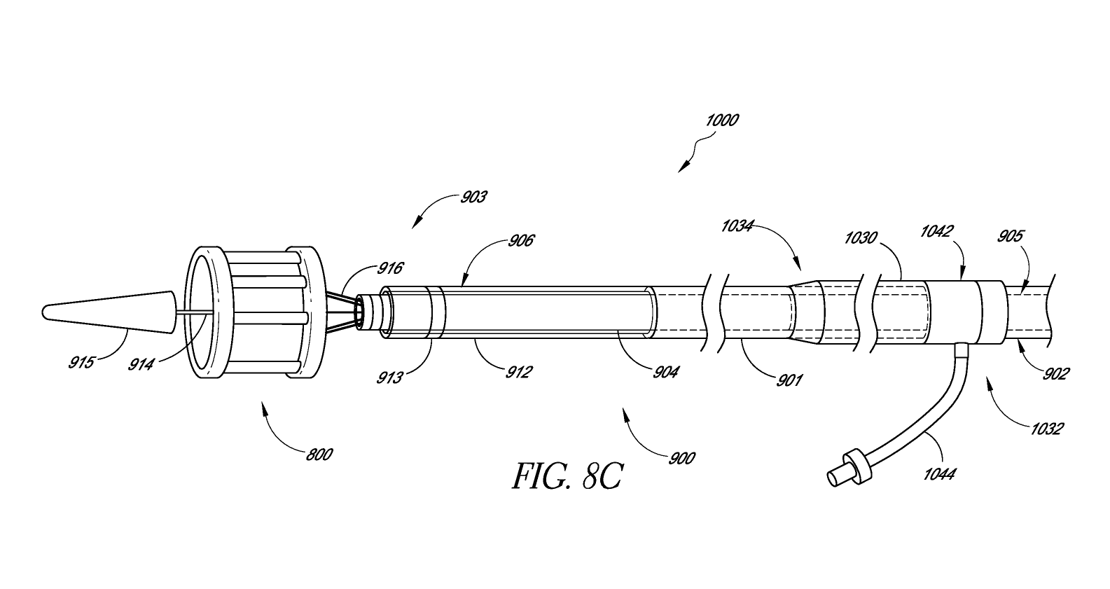

FIG. 8A illustrates an exemplary embodiment of a combined delivery system 1000 that can be used to deliver an implant 800, such as the implant embodiments described above. The combined delivery system 1000 can include an introducer catheter 1030 and that is positioned at least partially over the delivery catheter 900 described above. As will be explained in more detail below, in certain arrangements, it is advantageous to use the combined delivery system 1000 because the introducer catheter 1030 can have a smaller diameter than would possible if the introducer catheter 1030 and the delivery catheter 900 are separately introduced into the patient. For example, in the illustrated embodiment, the sheath jacket 912 of the delivery catheter 900 can have an outer diameter that is too large to be inserted through the introducer catheter 1030 (i.e., the outer diameter of the sheath jacket 912 can be larger than the inner diameter of the introducer catheter 1030 and in some embodiments the outer diameter of the sheath jacket 912 can be the same or substantially the same as the outer diameter of the introduce catheter). Accordingly, by preassembling or building the introducer catheter 1030 over a proximal portion of the delivery catheter 900, a reduced diameter combined delivery system 1000 can be created. In one embodiment, the introducer catheter 1030 is a 16 French introducer catheter capable of receiving a 16 French catheter. The outer diameter the sheath jacket 912 of the delivery catheter 900 and a distal end of the introducer catheter 1030 can be about 18 French or smaller. It is believed that such a combined delivery system 1000 has a smaller outer diameter than any known approved delivery system and introducer systems for transcatheter heart valves. The smaller delivery system size can reduce vascular complications such as aortic dissection, access site or access related vascular and/or distal embolization from a vascular source particularly in situations in which the patient's femoral artery has a smaller diameter.

FIG. 9 illustrates the introducer catheter 1030 of the illustrated embodiment in more detail. In general, the introducer catheter 1030 can comprise an elongate catheter having a proximal end 1032 and a distal end 1034. In some embodiments, the distal end 1034 of the introducer catheter 1030 can be tapered. The introducer catheter 1030 can comprise a seal assembly 1042 positioned at the proximal end 1032 of the introducer catheter 1030.

An inner diameter of the introducer catheter 1030 can be smaller than an outer diameter of a distal portion of the delivery catheter 900. In some embodiments, the inner diameter of the introducer catheter 1030 is about 16 French or less. In some embodiments, the introducer catheter 1030 can comprise a commercially available introducer catheter having an appropriate diameter. For example, in some embodiments, the introducer catheter 1030 is a 16 F introducer catheter commercially available from Cook Medical®.

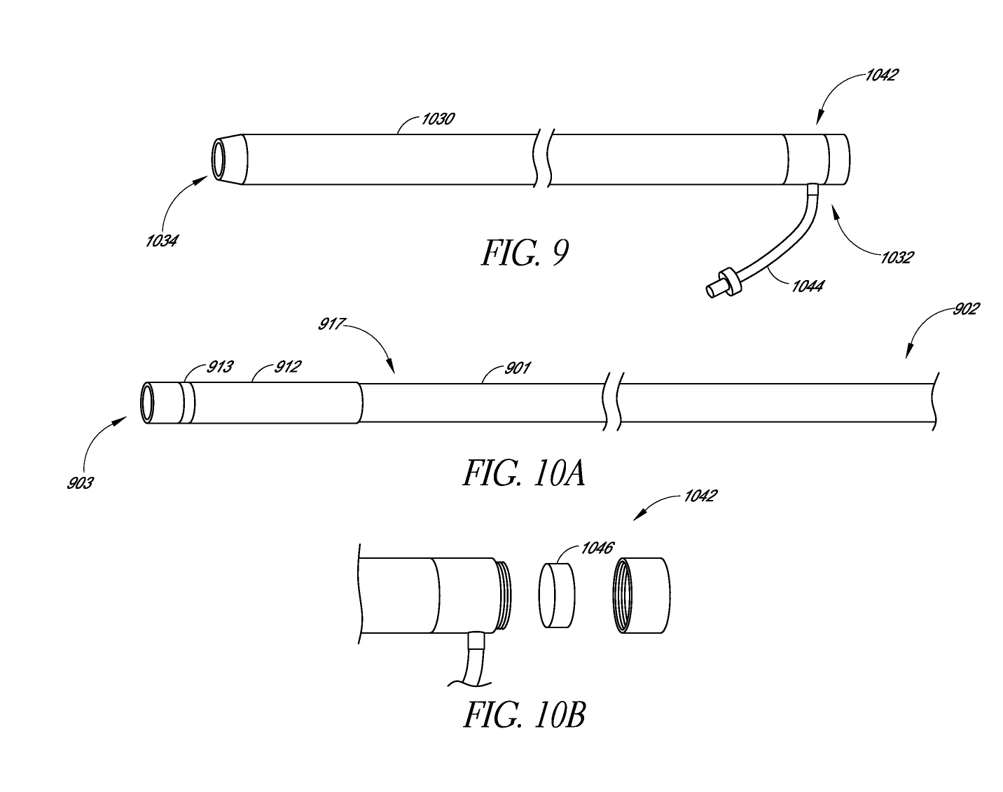

The seal assembly 1042 (see FIG. 10B) can threadably engage the proximal end 1032 of the introducer catheter 1030. The seal assembly 1042 can include a seal member 1046 configured to form a seal around the delivery catheter 900. The seal assembly 1042 can be adjusted to maintain the position of the introducer catheter 1030 relative to the delivery catheter 900 during the procedure. In some embodiments, the seal assembly 1042 comprises a hemostasis seal/valve configured to minimize blood loss during percutaneous procedures. In some embodiments, the seal assembly 1042 comprises a flush port 1044.

As discussed above, in general, the combined delivery system 1000 comprises the delivery catheter 900, which extends through the introducer catheter 1030. In the illustrated embodiment, the components of the delivery catheter 900 can be the same, similar, or identical to the corresponding components of the low crossing profile delivery catheter 900 discussed above accordingly. Accordingly, for the sake of brevity only certain components of the delivery catheter 900 will be described below.

As noted above, the delivery catheter 900 can include outer tubular member 901 having a proximal end 902 and a distal end 903, and an inner tubular member 904 also having a proximal end 905 and a distal end 906. The inner tubular member 904 extends generally through the outer tubular member 901, such that the proximal and distal ends 902, 903 of the inner tubular member 904 extend generally past the proximal end 902 and distal end 903 of the outer tubular member 901. In some embodiments, the delivery catheter 900 extends generally through the introducer catheter 1030, such that the proximal end 902 and the distal end 903 of the delivery catheter 900 extend generally past the proximal end 1032 and the distal end 1034 of the introducer catheter 1030.

In several embodiments, the outer diameter of the distal portion of the delivery catheter 900 and in particular, the sheath jacket 912, is larger than an inner diameter at the distal end of the introducer catheter 1030. In some embodiments, the outer diameter of the delivery catheter 900 is about 18 French or less, particularly at the distal portion of the delivery catheter 900. In some embodiments, the outer diameter at the proximal portion of the delivery catheter 900 is about 16 French or less. In FIGS. 8A and 8B, the outer diameter of the sheath jacket 912, the proximal portion of the guidewire tip 915 and the introducer catheter 1030 are illustrated as having different outer diameters. However, in certain arrangements, the outer diameters of these components 912, 915 and 1030 can be the same or substantially the same and the outer tubular member 901 can have a smaller outer diameter than these components. In certain arrangements, the sheath jacket 912 and the proximal portion of the guidewire tip 915 can have the same outer diameter or substantially same outer diameter as the proximal portions of the introducer catheter 1030.

FIG. 10 illustrates a closer view of the outer tubular member 901. The distal end 903 of the outer tubular member 901 can form the sheath jacket 912. As noted above, the sheath jacket 912 can house the implant 800 in a retracted state for delivery to the implantation site. In some embodiments, an outer diameter of the sheath jacket 912 is larger than an outer diameter of stem portion 917 of the outer tubular member 901. In the illustrated embodiment, the outer diameter of the sheath jacket 912 is larger than the inner diameter of at the distal end of the introducer catheter 1030 while the stem portion 912 has an outer diameter that is smaller than the inner diameter of the introducer catheter 1030. In some embodiments, the outer diameter of the sheath jacket 912 is about 18 F or less. In some embodiments, the outer diameter of the stem portion 917 of the outer tubular member 901 is 16 F or less. As described above, in some embodiments, the sheath jacket 912 is a separate component connected to the step portion 917 of the outer tubular member 901, while in other embodiments, the sheath jacket 912 is integrally formed with the proximal of the outer tubular member 901.

As explained above, in some arrangements, it can be advantageous to use the combined delivery system 1000 to reduce the diameter of the introducer catheter 1030 used to deliver the delivery catheter 900 to a treatment site. If the introducer catheter 1030 and delivery catheter 900 are separately introduced, the inner diameter of the introducer catheter 1030 has to be greater than the outer diameter of the largest portion of the delivery catheter 900 to be introduced into the patient. In contrast, in several embodiments of the combined delivery system 1000, the outer diameter of the distal portion of the delivery catheter 900 is greater than the inner diameter of the introducer catheter 1030. For example, in some embodiments, the outer diameter of the distal portion of the delivery catheter 900 is about 18 French, and the outer diameter of the proximal portion of the delivery catheter 900 is about 16 French. In some embodiments, the inner diameter of the introducer catheter 1030 is about 16 French. In some embodiments, the introducer catheter 1030 can be pre-installed over the proximal portion of the delivery catheter 900.

### Method of Deployment Using the Combined Delivery System

In several embodiments, an implant 800 may be deployed in an aortic position using the combined delivery system 1000 described above and a minimally invasive procedure. In some embodiments, the method generally comprises gaining access to the aorta, most often through the femoral artery. The vascular access site can be prepared according to standard practice, and the guidewire can be inserted into the left ventricle through the vascular access.

As shown in FIG. 8A and as described above, the introducer catheter 1030 can be pre-installed over the delivery catheter 900 prior to performing the minimally invasive procedure. For example, the manufacturer can pre-install the introducer catheter 1030 over the delivery catheter 900. In some embodiments, the manufacturer extends the delivery catheter 900 through the introducer catheter 1030 prior to completing assembly of the combined delivery system 1000. For example, in some arrangements, it can be desirable to extend the delivery catheter 900 through the introducer catheter 1030 prior to attaching a handle to the proximal end 902 of outer tubular member 901. In other arrangements, it can be desirable to extend the delivery catheter 900 through the introducer catheter prior to attaching the sheath jacket 912 or implant 800 to the distal end 940 of the delivery catheter 900.

In other embodiments, the operator (e.g., a nurse, physician, or other individual) extends the delivery catheter 900 through the introducer catheter 1030 prior to inserting the introducer catheter 1030 or delivery catheter 900 into the patient. In some embodiments, the handle of the outer tubular member 901 can be removable, thus allowing the user to remove the handle and extend the delivery catheter 900 through the introducer catheter 1030 prior to inserting the introducer catheter 1030 or delivery catheter 900 into the patient.

In some embodiments, after the manufacturer or operator extends the delivery catheter 900 through the introducer catheter 1030, a distal portion of the delivery catheter 900 extends distally from the distal end 1034 of the introducer catheter 1030. In some embodiments, the distal sheath jacket 912 or implant 800 extends distally from the distal end 1034 of the introducer catheter 1030.

After the combined delivery system 1000 is assembled, as shown in FIG. 10, the combined delivery system 1000 carrying the cardiovascular prosthetic implant 800 can be translumenally advanced. In some embodiments, the combined delivery system 1000 is inserted over the guidewire. In such embodiments, the guidewire tip 915 can be inserted directly into the access vessel over the guidewire such that the guidewire tip dilates the access vessel for the introducer catheter 1030. In some embodiments, the combined delivery system 1000 is advanced until the seal assembly 1042 reaches the patient. In other embodiments, the introducer catheter 1030 is held in place while the delivery catheter 900 is further advanced as shown in FIG. 8B. The delivery catheter 900 can be advanced to a position proximate a native valve. In other embodiments, the entire combined delivery system 1000, including both the introducer catheter 1030 and the delivery catheter 900 can be advanced to a position proximate a native valve.

After the delivery catheter 900 is advanced over the aortic arch and past the aortic valve, the position of the outer tubular member 901 relative to the introducer catheter 1030 can be maintained by adjusting the seal assembly 1042 to form a seal around the outer tubular member 901.

As shown in FIG. 8C, in some embodiments, the implant 800 can be revealed or exposed by retracting the outer tubular member 901 partially or completely while holding the inner tubular member 904 stationary and allowing proper placement at or beneath the native valve. In some embodiments, the implant can also be revealed by pushing the inner tubular member 904 distally while holding the outer tubular member 901 stationary. Once the implant 800 is unsheathed, it may be moved proximally or distally, and the fluid or inflation media may be introduced to the cuff 802 providing shape and structural integrity. In some embodiments, the distal toroid of the inflatable cuff or inflatable structure is inflated first with a first liquid, and the implant 800 is positioned at the implantation cite using the links between the implant 800 and the combined delivery system 1000. In some embodiments, no more than three links are present. In some embodiments, the links are PRL tubes 916, which can be used to both control the implant 800 and to fill the inflatable cuff. The implant 800 may be otherwise inflated or controlled using any of the other methods disclosed above.

In some embodiments, the links are PRL tubes 916, which can be used to both control the implant 800 and to fill the inflatable cuff.

The deployment of the implant 800 can be controlled by the PFL tubes 916 that are detachably coupled to the implant 800. The PFL tubes 916 are attached to the cuff 802 of the implant 800 so that the implant 800 can be controlled and positioned after it is removed from the sheath or delivery catheter 900. Preferably, three PFL tubes 916 are used, which can provide precise control of the implant 800 PFL tubes 916 during deployment and positioning. The PFL tubes 916 can be used to move the implant 800 proximally and distally, or to tilt the implant 800 and change its angle relative to the native anatomy.

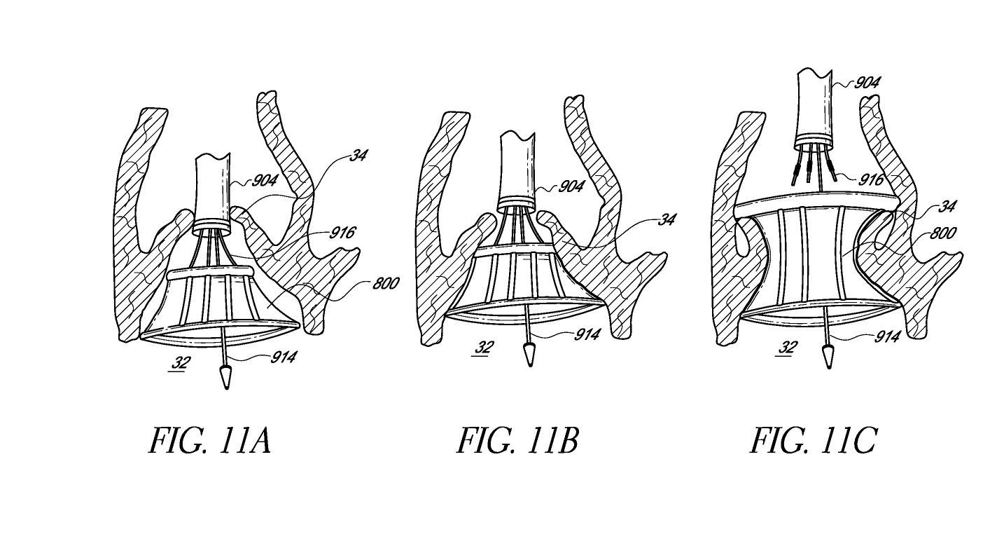

In some embodiments, the implant 800 contains multiple inflation valves 810 to allow the operator to inflate specific areas of the implant 800 with different amounts of a first fluid or a first gas. With reference to FIGS. 11A-C, in some embodiments, the implant 800 is initially deployed partially in the ventricle 32 (FIG. 11A). The inflation channel 808 is filled partially, allowing the distal portion of the implant 800 to open to approximately its full diameter. The implant is then pulled back into position at or near the native valve 34 annulus (FIG. 11B). In some embodiments, the distal toroid 807b is at least partially inflated first, and the cardiovascular prosthetic implant 800 is then retracted proximally for positioning the cuff across the native valve 34. The distal ring 807b seats on the ventricular side of the aortic annulus, and the implant 800 itself is placed just above the native valve 34 annulus in the aortic root. At this time, the PFL tubes 916 may act to help separate fused commissures by the same mechanism a cutting balloon can crack fibrous or calcified lesions. Additional inflation fluid or gas may be added to inflate the implant 800 fully, such that the implant 800 extends across the native valve annulus extending slightly to either side (See FIG. 11C). The PFL tubes 916 provide a mechanism for force transmission between the handle of the deployment catheter 900 and the implant 800. By moving all of the PFL tubes 916 together or the inner tubular member 904, the implant 800 can be advanced or retracted in a proximal or distal direction. By advancing only a portion of the PFL tubes 916 relative to the other PFL tubes 916, the angle or orientation of the implant 800 can be adjusted relative to the native anatomy. Radiopaque markers on the implant 800 or on the PFL tubes 916, or the radio-opacity of the PFL tubes 916 themselves, can help to indicate the orientation of the implant 800 as the operator positions and orients the implant 800.

In some embodiments, the implant 800 has two inflation valves 810 at each end of the inflation channel 808 and a check valve 811 in the inflation channel 808. The check valve 811 is positioned so the fluid or gas can flow in the direction from the proximal toroid 807a to the distal toroid 807b. In some embodiments, the implant 800 is fully inflated by pressurizing the endoflator attached to the first PFL tube 916 that is in communication with the first inflation valve 810 that leads to the proximal toroid 807a, while the endoflator attached to the second inflation valve 810 that is in communication with the distal toroid 807b is closed. The fluid or gas can flow into the distal toroid 807b through the one-way check valve. The proximal toroid 807a is then deflated by de-pressurizing the endoflator attached to the second inflation valve. The distal toroid 807b will remain inflated because the fluid or gas cannot escape through the check valve 811. The implant 800 can then be positioned across the native annulus. Once in the satisfactory placement, the proximal toroid 807a can then be inflated again.

In some embodiments, the implant 800 may only have one inflation valve. When the inflation channel 808 is inflated with the first fluid or gas, the proximal portion of the implant 800 may be slightly restricted by the spacing among the PFL tubes 916 while the distal portion expands more fully. In general, the amount that the PFL tubes 916 restricts the diameter of the proximal end of the implant 800 depends on the length of the PFL tubes 916 extend past the outer tubular member 901, which can be adjusted by the operator. In other embodiments, burst discs or flow restrictors are used to control the inflation of the proximal portion of the implant 800.

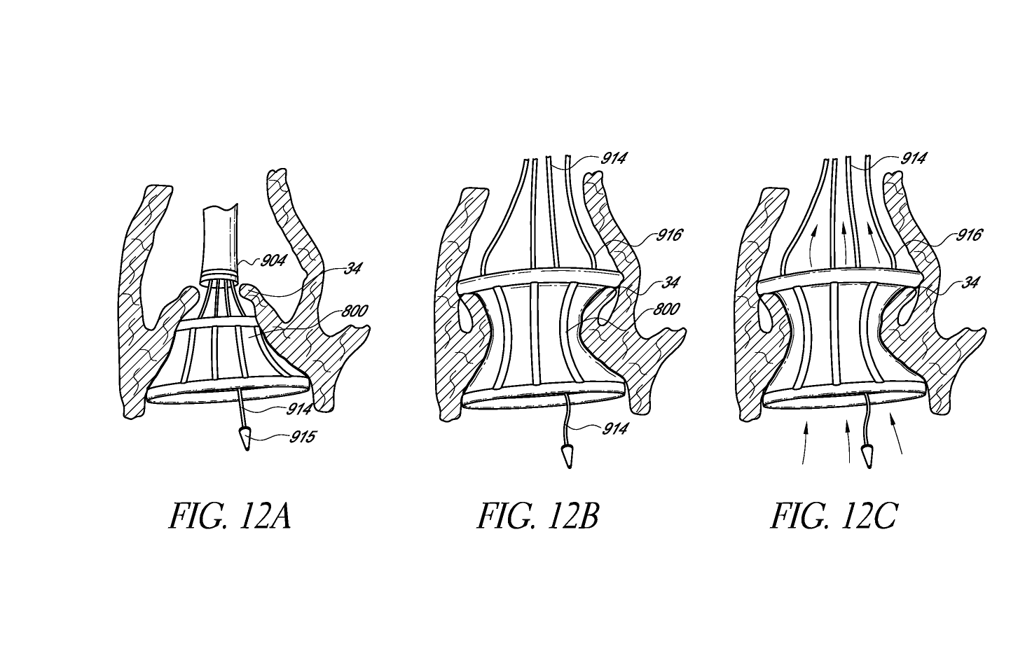

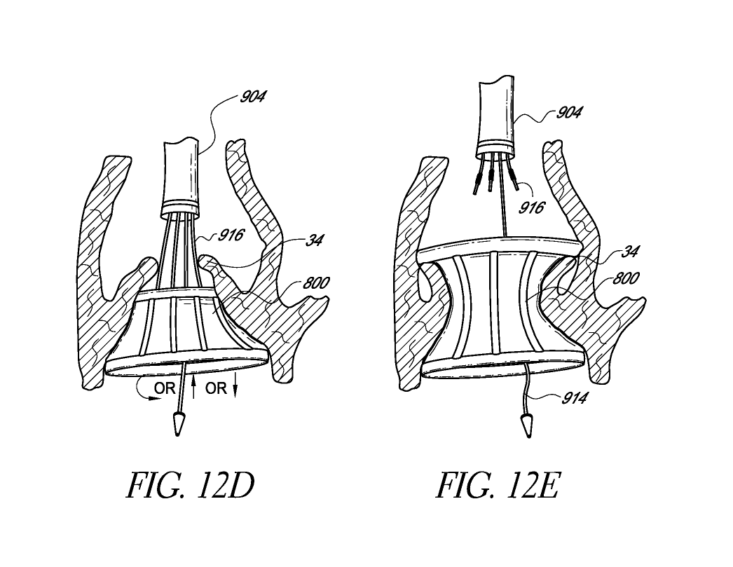

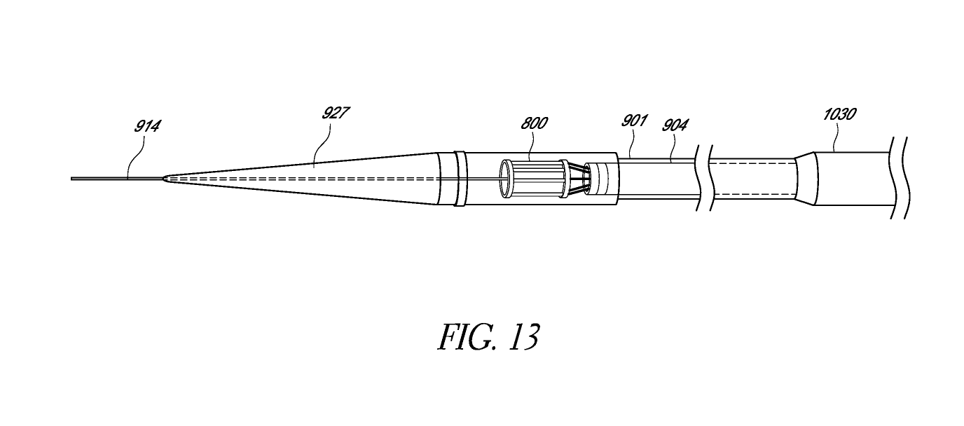

The implant 800 can also be deflated or partially deflated for further adjustment after the initial deployment. As shown in FIG. 12A, the implant 800 can be partially deployed and the PFL tubes 916 used to seat the implant 800 against the native aortic valve 34. The implant 800 can then be fully deployed as in shown in FIG. 12B and then tested as shown in FIG. 13C. If justified by the test, the implant 800 can be deflated and moved as shown in FIG. 12D to a more optimum position. The implant 800 can then be fully deployed and released from the control wires as shown in FIG. 12E.

As discussed above, in some embodiments, the first inflation fluid or gas can be displaced by an inflation media that can harden to form a more permanent support structure in vivo. Once the operator is satisfied with the position of the implant 800, the PFL tubes 916 are then disconnected, and the catheter is withdrawn leaving the implant 800 behind (see FIG. 12C), along with the hardenable inflation media. The inflation media is allowed to solidify within the inflatable cuff. The disconnection method may included cutting the attachments, rotating screws, withdrawing or shearing pins, mechanically decoupling interlocked components, electrically separating a fuse joint, removing a trapped cylinder from a tube, fracturing a engineered zone, removing a colleting mechanism to expose a mechanical joint or many other techniques known in the industry. In modified embodiments, these steps may be reversed or their order modified if desired.

In some arrangements, it may be desirable to deliver a cardiovascular prosthetic implant 800 using a combined delivery system 1000 to reduce the number of components and steps necessary to position the cardiovascular prosthetic implant 800. For example, if the introducer catheter is inserted separately from the delivery catheter, the operator uses a dilator to facilitate delivery of the introducer catheter. In some scenarios, the dilator includes a flexible, elongate catheter body and a generally cone-shaped tip. The dilator is often a separate component that extends through the introducer catheter and must be removed after the introducer catheter is delivered to the appropriate position. After the dilator is removed, the operator inserts the delivery catheter through the introducer catheter. It can be advantageous to eliminate the use of the dilator or eliminate the catheter exchange step by delivering the cardiovascular prosthetic implant 800 using a combined delivery system 1000. Instead of relying on the separate dilator component, the combined delivery system 1000 can use the guidewire tip 915 to function as the dilator. As described above, in some embodiments, the guidewire tip 915 can be cone-shaped, bullet-shaped, or hemispherical-shaped to facilitate dilation. Further, the diameter of the guidewire tip 915 can be configured to form a smooth transition from the distal end of the sheath jacket 912 to the guidewire tip 915. The smooth transition can help prevent the distal end of the introducer catheter 1030 from damaging a vessel wall.

In certain arrangements, it is advantageous to deliver a cardiovascular prosthetic implant 800 using a combined delivery system 1000 to reduce the number steps necessary to remove the combined delivery system 1000 after the implant 800 is delivered to the appropriate location. For example, if the introducer catheter is inserted separately from the delivery catheter, the delivery catheter can be completely removed from the patient before the introducer catheter is removed from the patient. In some scenarios, it can be desirable to remove both the introducer catheter and delivery catheter simultaneously using the combined delivery system 1000. After the implant 800 is delivered to the appropriate location, the PFL tubing 916 can be retracted proximally into the inner tubular member 904. In some embodiments, the delivery catheter 900 is retracted proximally until a proximal end of the sheath jacket 912 abuts the distal end 1034 of the introducer catheter 1030. The guidewire tubing 914 can be retracted proximally until the guidewire tip 915 closes the distal end of the outer tubular member 901 and forms a smooth transition from the distal end 1034 of the introducer catheter 1030 to the guidewire tip 915. The smooth transition can help prevent the distal end 1034 of the introducer catheter 1030 from damaging the blood vessel as the introducer catheter is removed from the patient. The introducer catheter 1030 and the delivery catheter 900 can then be removed from the patient simultaneously.

With the integral introducer, it is desirable to have a relatively long tapered tip to facilitate introduction through tortuous arteries and tensioning of the sutures for arterial closure upon device removal, but for safe deployment in the relatively small ventricle it is desirable to have a tip that does not take up too much space. Several embodiments addressing this issue are described. These embodiments can be used in combination with the various embodiments described above.

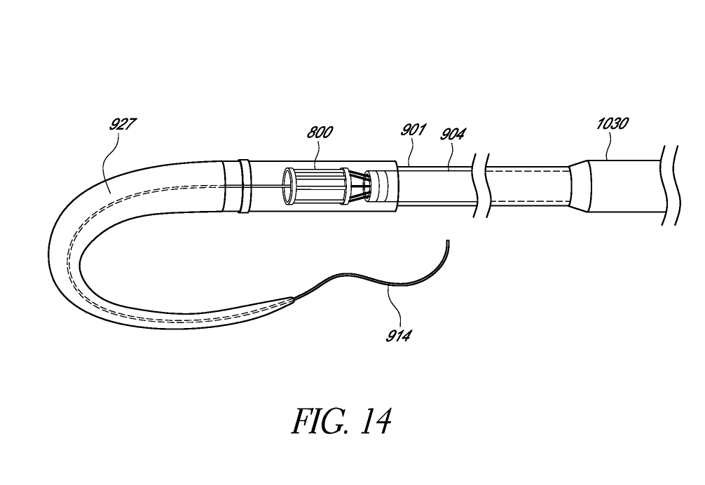

In a first embodiment shown in FIG. 13, the distal portion of the catheter tip 927 can be about 2 to 8 cm, similar to a dilator introducer for a similarly sized introducer, but is extremely flexible, so that it can follow the curve of the guidewire 914 inside the ventricle (see e.g., FIG. 14). In one embodiment the tip is manufactured from a material such as silicone or urethane with a durometer of less than about 25 A. In another embodiment the outer surface of the tip 927 is substantially continuous but material from the internal volume of the tip is omitted allowing the tip to flex. Preferably the tip 927 is capable of bending to a radius of less than 3 cm with less than 1 lb force. More preferably the tip 927 is capable of bending to a radius of less than 3 cm with less than 0.5 lb force. In another embodiment the tip 927 has a preset curve with a radius of approximately 2 to 8 cm or more preferably about 3 to 5 cm. Preferably the curved tip 927 is substantially straightened when placed over the stiff section of a very stiff 0.035 guidewire 914, and returns to a curved shape over the flexible or curved distal section of the guidewire 914. Preferably the tip 927 is radiopaque. This can be accomplished by filling the tip 927 with a radiopaque material such as barium sulfate, tungsten or tantalum.

In another embodiment the device has a long tip in one configuration and a short tip in a second configuration, where the long tip is greater than about 3 cm and the short tip is less than about 3 cm. In a similar embodiment the long tip is greater than about 2 cm and the short tip is less than about 2 cm. The device is advanced through the iliac arteries in the long tip configuration and advanced near the treatment location into the ventricle in the short tip configuration. In one embodiment a long tip fits over a short tip and is held in place by at least one tension member which extends to a proximal portion of the device. After the device has passed through the challenging access site the tension members are loosened allowing the long tip to move away form the short tip, but containing it for later removal.

In another embodiment the tip has a straight configuration and a bent configuration and can be oriented from one configuration to the other by devices of a mechanism such as a pullwire.

In another embodiment the tip is inflatable, achieving a long configuration when pressurized and a short configuration when deflated, or when a vacuum is applied.

When treating a patient with the integral introducer sheath it is typically to introduce the device with the guidewire already in position across the aortic valve. In some cases this can present a challenge or risk to keep the guidewire in proper position during device insertion. The embodiments describe herein include several methods to facilitate crossing the native valve with the guidewire after the device is inserted

In one embodiment the guidewire exits the distal tip of the guidewire at an angle at least 5 degrees from the axis of the delivery system, and preferably between 10 and 40 degrees. This allows the delivery catheter to be rotated to point the guidewire directly at the aortic valve to allow easy crossing of the valve with the guidewire. In one embodiment the shape of the tip is similar to the shape of a coronary guide catheter commonly used to cross the aortic valve.

In another embodiment the tip is deflectable and the bend of the tip can be selected by the operator. In one embodiment this is accomplished by use of a pull wire.

One embodiment includes a steerable guidewire as an accessory. Steerable guidewires are commonly known in the art.

In another embodiment a lumen is provided with a bend near the distal end and an outside diameter of approximately 0.035 or configured so that it passes through the guidewire lumen. The inside diameter of the lumen is configured so that a 0.032, 0.018 or 0.014 or 0.009 guidewire can pass through it. This additional lumen can be used to control the guidewire and facilitate crossing the aortic valve with the guidewire.

When treating a patient with the integral introducer sheath it is typically necessary to introduce the device with the guidewire already in position across the aortic valve. In some cases this can present a challenge or risk to keep the guidewire in proper position during device insertion. The embodiments described herein include several methods to minimize the difficulty and risk of the sheath exchange.

In one embodiment the guidewire lumen exits the catheter at least 5, 10 20 or 50 cm distal to the proximal end of the catheter. This allows a single operator to control the guidewire position during the removal of the smaller sheath and the insertion of the device. I

In one embodiment the guidewire passes through a lumen in the tip, where one end of the lumen is at approximately the distal end of the tip and the second end of the lumen is near a side of the tip distal to where the tip is in contact with the sheath portion of the delivery catheter. This provides the benefits of single operator guidewire control while additionally allowing the connection to the tip to be of smaller cross sectional area, allowing for further profile reduction.

When treating a patient with the integral introducer sheath it may be desirable to have a larger diameter sheath for certain manipulations that are not used in all procedures, such as retrieval of the implant. In some embodiments the introducer can expand in these situations but maintains the low profile of the device during normal use. The expandable introducer may be of a design similar to the e-sheath marketed by Edwards Lifesciences or of a design similar to one marketed by onset medical. In another embodiment the introducer sheath can be made from a polymer in a tubular cross section that expands during retrieval through elastic and plastic deformation. The expanded configuration is preferably at least 10 percent larger than the non expanded configuration. The ID of the expanded configuration is preferably similar to the OD of the non expanded configuration. The ID of the expanded configuration is preferably larger than the OD of the non expanded configuration.

For the withdrawal of the device with the integral sheath, especially when used with percutaneous closure techniques utilizing device such as prostar or proglide marketed by Abbot laboratories, it is preferable to be able to tighten the sutures on the tapered tip of the device as the device is being removed from the patient. To facilitate easy removal the preferred embodiments have a mechanism to lock the tip to the catheter body and or the catheter body to the introducer sheath, so that by pulling back on a single component while cinching the sutures is a simple procedure requiring a minimum of coordination between multiple operators.

In one embodiment the tip and the largest diameter portion of the outer sheath are collapsible to facilitate their removal through an integral introducer that is not substantially expandable. In one embodiment the components are mechanically collapsible such that by providing axial force to pull the components into the introducer sheath they collapse. In one embodiment the tip is made from nylon 12 with a hollow cross section and a wall thickness of between 0.005 and 0.050 in.

In one embodiment the lock mechanism is a cam located in the proximal handle that locks the guidewire lumen to the catheter body, substantially preventing relative motion between the catheter body and the tip. In another embodiment a lock mechanism is a toughy-borst type valve located on the proximal end of the integral introducer sheath that can be tightened to prevent relative motion between the integral introducer sheath and the catheter body.

For the withdrawal of the device with the integral sheath, especially when used with percutaneous closure techniques utilizing device such as prostar or proglide marketed by Abbot laboratories, it is important to know the relative location of the tip, the distal and proximal ends of the large diameter portion of the delivery device and the distal portion of the integral introducer sheath.

One embodiment of the device includes radiopaque markers at the locations described above. In another embodiment a visible mark on the outer portion of the delivery device that when aligned with a visible mark or edge of the bub of the integral introducer, indicates that the proximal end of the large diameter portion of the delivery device is aligned with the distal end of the delivery catheter.

One embodiment includes an accessory device for accessing difficult iliac anatomies. An inverted tip balloon is inserted though the contralateral side, and advanced through the aortic bifurcation back into the access vessel. The inverted tip allows the guidewire to be advanced through the device, and then through the guidewire lumen of the inverted tip balloon. The balloon can be advanced close to the device so that the tip of the device is inside the inverted tip of the balloon. the device can be advanced through severe calcification and tortuosity by inflating the balloon and advancing the system with the balloon. The inverted tip balloon has an OD similar to the OD of the delivery system, preferably between 3 mm and 8 mm. The balloon has a rated burst pressure between 2 and 20 atmospheres and preferably a guidewire lumen of approximately 0.036 in diameter. The balloon preferably has low compliance to maintain the inverted tip shape at pressure and allow dilation of the vessel to the size needed for device delivery without causing unnecessary trauma.

Deployment System

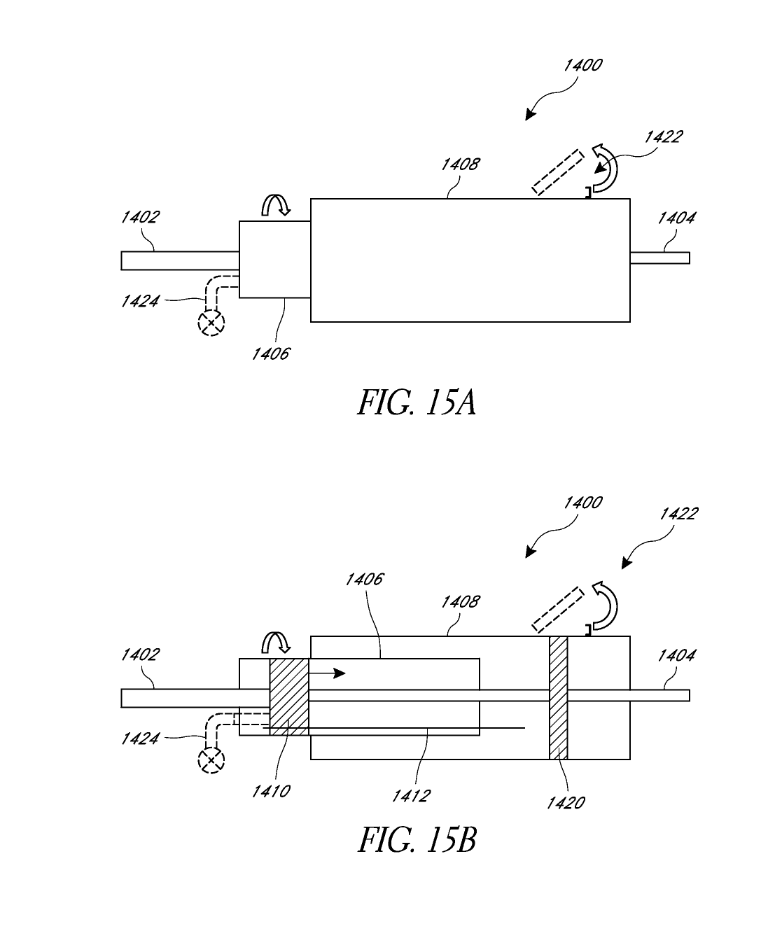

FIGS. 15A and 15B are schematic side and cross-sectional illustrations of a deployment system 1400 that can be used to move (e.g., retract) a first member 1402 with respect to a second member 1404. In one arrangement, the first member can be 1402 can be an outer member (e.g., an outer sheath or tube of a catheter) while the second member 1404 can be an inner member (e.g., an inner catheter or tube of a catheter).

In one arrangement, the system 1400 can include a rotational member 1406 and a handle 1408. The rotational member 1406 can have an actuator 1407 that can extend outside the handle 1408 such that a user can rotate the rotational member 1406 with respect to the handle 1408. For example, in one embodiment, the handle 1408 can be grasped with one hand while the actuator 1407 is rotated with another hand. In an embodiment, the handle 1408 and actuator 1407 can be configured for being held and actuated by one hand, for example, by providing a dial or wheel positioned near a thumb of a user grasping the handle 1508. In the illustrated embodiment, the actuator 1407 is positioned on a distal end of the handle but in other arrangements the actuator can be positioned partially or wholly within the length of the handle and/or at a proximal end of the handle.

As shown in FIG. 15B, the first member 1402 can extend into the rotational member 1406 and can be coupled to a carriage 1410 positioned within the rotational member 1406. An inner surface of the rotation member 1406 can include internal or external threads or thread like members that interact with corresponding internal and/or external protrusions or grooves on the carriage 1410. An alignment member 1412 that is coupled to the handle 1408 can extend into the rotational member 1406 to limit rotation of the carriage 1410 within the rotational member 1406.

In use, a user can rotate the rotational member 1406 (e.g., by grasping a portion of the rotational member or an actuator 1407 coupled thereto) that extends beyond, through and/or is exposed through a portion of the handle. As the rotational member 1406 is rotated within the handle 1408 (which can remain stationary with respect to the rotational member 1406), the carriage 1410 can ride along the internal and/or external thread or thread-like members and can travel the longitudinal length of the rotational member 1406 or a portion thereof as rotational movement of the carriage is limited by the alignment member 1412. The carriage 1410, in turn, can be coupled to the first member 1402 such that the first member 1402 is retracted as the carriage 1410 moves proximally within the rotation member 1406. An advantage of the illustrated arrangement is that the carriage 1410 can move at least partially within a portion of the rotational member 1406 that is actuated by the user. This arrangement results in a compact configuration of the system 1400.

The second member 1404 can be coupled to the handle 1408 (as described below) and can extend through the carriage 1410 and the first member 1402 such that movement of the carriage 1410 within the handle 1408 will cause relative movement of the first member 1402 with respect to the second member 1404.

The illustrated deployment system 1400 can also include a releasable coupling mechanism 1420. As show in FIG. 15B, the second member 1404 can extend through (or partially through) the releasable coupling member 1420 which, in turn, can be coupled to the handle 1408. When in a “locked” position (e.g., illustrated by solid lines in FIGS. 15A and 15B), movement between the second member 1404, the coupling member 1420 and the handle 1408 is limited. Accordingly, during the movement described above, the first member 1402 can move while the second member 1404 remains stationary (or substantially stationary) with respect to the handle 1408. The coupling mechanism 1420 can include an actuator 1422 (e.g., a lever, knob, etc.) that can move the coupling mechanism 1420 from a locked to an unlocked position (e.g., illustrated by solid and dashed lines in FIGS. 15A and 15B). In the unlocked position (e.g., the dashed line position), the second member 1404 can be released from the coupling member 1420 such that the second member 1404 can be removed from the handle 1408 (or vice versa). In one arrangement, this can allow the first member and handle to be withdrawn over the second member 1404 such that the first member 1402 can be removed from the patient. A third member (e.g., a retrieval catheter) can then be inserted over the second member 1404 which can remain in the patient.

As shown in FIGS. 15A and 15B, a flush port 1424 can be coupled to the carriage 1410 such that movement of the carriage 1410 can cause movement of the flush port 1424. The flush port 1424 can be used to deliver a flushing material (e.g., liquid) to components within the system 1400 such as the handle 1409, the carriage 1410, and/or the first and second members 1402, 1404.

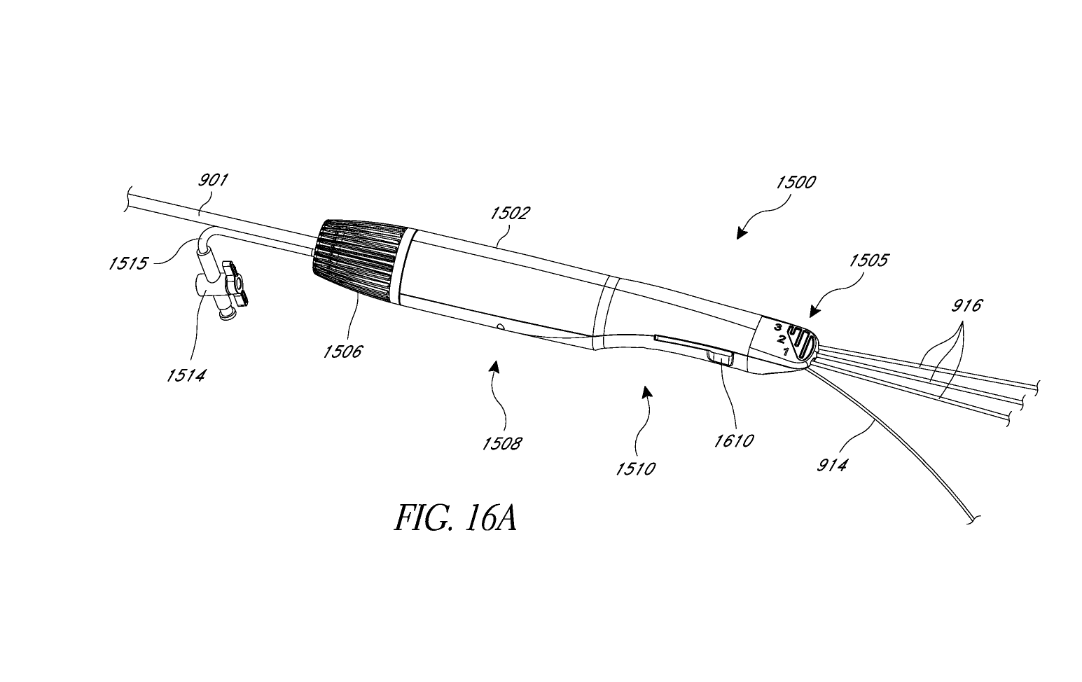

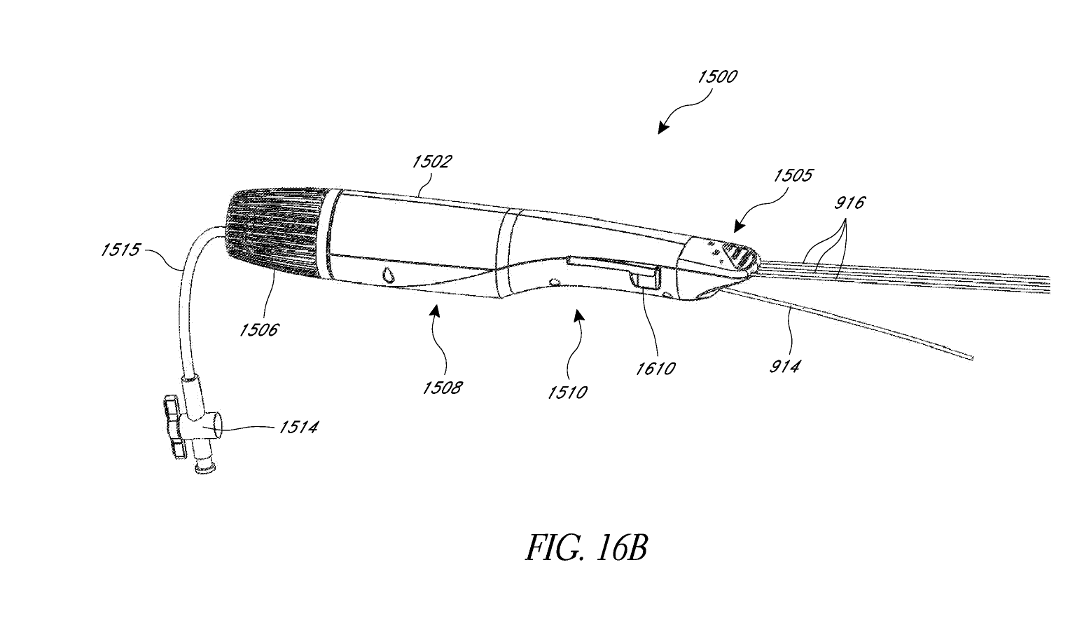

FIG. 16A is a side perspective view of a deployment system 1500 that has certain features in common with the system 1400 illustrated schematically in FIGS. 15A and 15B. The illustrated deployment system 1500 can be used in combination with the deployment catheter 900 embodiments described above and/or with modified arrangements of such embodiments and/or sub-combinations thereof. As described below, in the illustrated embodiment, the deployment system 1500 can replace the inner and outer sheath handles 907, 908 (described above). As will be described below, in an embodiment, the deployment system 1500 can be used to retract the outer sheath 901 with respect to the inner sheath (e.g., an inner tubular member) 904 with certain advantages as compared to the inner and outer sheath handles 907, 908. FIGS. 16B-31 provide additional views of the deployment system 1500 and of various components of the deployment system 1500.

While the deployment system 1500 will be described and illustrated in combination with certain features of the deployment catheter 900 and implant 800 described herein, features of the deployment system 1500 can also be used independently of the embodiments described herein and can have advantages in other types of deployment catheters and/or with other types of implants particularly in arrangements where a first component (e.g. a first catheter or tubular member) is retracted or moved relative to a second component (e.g., a second catheter or tubular member). For example, the deployment system can be used to retract an outer sheath relative to an inner member or inner tubular member. The deployment system 1500 can also be used independently or in combination with the introducer catheter 1030 and combined delivery system 1000 described above. In such combined arrangements, the introducer catheter 1030 can be preassembled or built over a proximal portion of the delivery catheter 900 as described above.

With initial reference to FIG. 16A, the deployment system 1500 in the illustrated embodiment comprises a handle or body 1502. The distal end 1504 of the handle 1502 can include a knob 1506, which, will as will be explained in detail below can be rotated or twisted in relative to the handle 1502 to retract the outer tubular member (sometimes referred to as “outer sheath”) 901 while holding the inner tubular member 904 (not shown in FIG. 16A) stationary or substantially stationary relative to the outer sheath 901. The proximal end 902 of the outer sheath 901 can be coupled to a portion the deployment system 1500 as explained below. A distal end 903 of the sheath can form a sheath jacket 912 as described above with the inner tubular member 904 extending through outer sheath 902 and into the deployment system 1500 as described below. The portions of the outer sheath 901 and inner tubular member 904 extending distally from the handle 1501 can be configured in accordance with the embodiments described above.

For example, as described above, the inner tubular member 904 can comprise a multi-lumen tube (see e.g., FIG. 6) that can include at least four lumens. One of the lumens can accommodate the guidewire tubing 914 and each of the other lumens can accommodate a positioning-and-fill lumen (PFL) tubings 916. The guidewire tubing 914 can be configured to receive a guidewire. Modified embodiments can include no, less or more lumens and/or lumens for different purposes or components.

As shown in FIGS. 15 and 16, the handle 1502 can have a generally cylindrical portion 1508 at its distal end adjacent the knob 1506 and recessed portion 1510 at the proximal end of the handle 1506. In an embodiment, the handle can have a different outer shape e.g., generally cylindrical, conical, peanut shaped, etc. The positioning-and-fill lumen (PFL) tubes 916 can extend from openings in the proximal end of the recessed portion 1510. As explained below, the tubes 916 can be individually retracted, rotated and/or pushed to provide control over the implant as described above. Markings, visual or physical indicia etc. 1505 can be provided on the handle 1502 adjacent the openings to provide labels to the tubes 916. For example, the illustrated embodiment includes the labels “1”, “2” and “3” and corresponding raised ridges of different lengths. Additionally and/or alternatively, the openings in the proximal end from which the tubes 916 extend can be non-coplanar. For example, the opening labeled “2” may be slightly elevated or lowered compared to the openings labeled “1” and “3”. Non-coplanar openings may allow a user to define the spacing between the tubes 916. The positioning of the openings can be selected to help minimize contact of the tube 916 with the handle 1506. The position of the openings can be chosen to optimize the exit path of the tube 916 and to reduce friction between the tube 916 and the handle 1506. An advantage of positioning the openings at different elevations is that the user can determine which tube 916 they are grasping without having to look at the tube 916. That is, in one embodiment the middle tube 916 is positioned lower or higher than the other two tubes 916. In such an arrangement, the user can feel that one tube (e.g., the center tube 916) is positioned at different elevation than the other tubes and thus feel without looking that they are grasping and/manipulating the middle tube 916. In a similar manner, the user can feel that they are grasping the tubes to the sides of the middle tube 916 by sensing the difference in elevation with their hands.

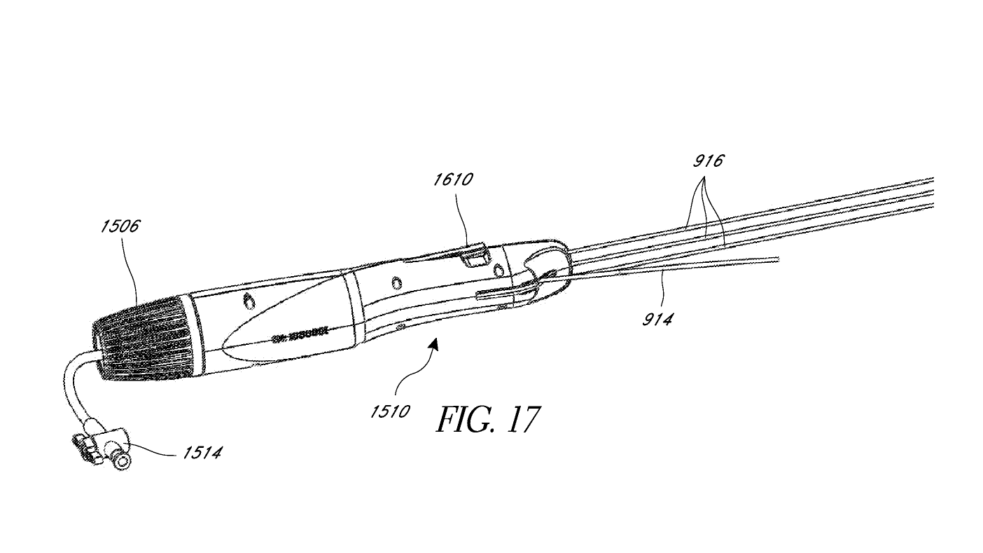

As seen in FIG. 17, the recessed portion 1510 of the handle 1502 can include a slot 1512 through which the guidewire tubing 914 can extend. This slot 1512 can have several advantages. For example, standard guidewires for aortic valve replacement procedures can be manufactured in various lengths, but typically 260 cm wires are used for aortic valve replacement procedures. These guidewires can have a relatively stiff section approximately 260 cm in length with a more flexible or floppy section at the distal end which is typically 1 to 10 cm in length. Using a substantially longer wire can be cumbersome because it can extend beyond the normal sterile field. However, using a substantially shorter wire can be impractical because to advance the device over the guidewire preferably the length of guidewire extending outside the patient's body is longer than the delivery device. This arrangement allows one area of the guidewire to be stabilized by a physician at all times as the device is being inserted. To maximize the length of the vascular path that can be treated with a given length guidewire, the length of the outer sheath is preferably be balanced with the length of the guidewire lumen, and it is desirable to minimize the length between the distal portion of the handle and the most proximal portion of the handle that the guidewire passes through. Applicant's illustrated solution for accomplishing this design goal is to provide the slot 1512 in the handle 1502 that allows the guidewire tubing 914 to exit the handle distal to the proximal end of the handle 1502.

In one embodiment the guidewire slot 1512 is at least 0.2, 0.4, 0.6, 0.8, 1.0, 1.2, 1.4, 1.6, 1.8 or, 2.0 inches in length or any value including and between 0.2 and 2.0 inches. In one embodiment, the guidewire slot 1512 is designed to allow the guidewire tubing 914 and guidewire to pass outside the physician's hand which is holding the handle in recessed portion. In such arrangements, portions of the fingers or hand the holding the handle 1502 in the recessed portion 1510 can lie between the guidewire tubing 914 and the handle 1502. In such embodiments, embodiment the guidewire slot 1512 can be at least 2.0, 3.0, 4.0, or 5.0 inches in length or any length including and between 2.0 and 5.0 inches.

Normally the length of vasculature that can be treated is defined by the following equation. Where:
A is the length of the device from most proximal portion that tracks over the guidewire to the most distal portion that tracks over the guidewire.
A′ is the length of the device that can be tracked through the introducer and into the patient anatomy measured from the distal tip of the device to the hub portion of the device larger than the entry port of the indicated introducer sheath.
B is the length of the stiff portion of the guidewire that is safe to track a device over, not including the floppy tip length that is to prevent damage to the native tissues by the guidewire.
C is the floppy tip length at the tip of the guidewire.
D is the length of the path from the hub of the introducer sheath to the target location in the patient anatomy where the distal tip of the device is intended to be tracked to.
L is the effective length of the handle defined as A-A′
L′ is the physical length of the handle defined as the portion of the handle where the diameter of the handle is greater than the inside diameter of the proximal portion of the introducer sheath the device is intended to be compatible with.

In one embodiment to allow adequate guidewire length to insert the device and maintain control of the guidewire while the device is advanced over the wire to the introducer hub:

B>/=D+A

To allow access to the target location with the delivery system the delivery system is preferably long enough

A′>/=D

In one embodiment, to maximize the length of the longest anatomic pathway that can be treated for a given standard length guidewire, L should be made as short as possible. In some instances, for ergonomic reasons or for packaging mechanisms required for handle function, it is desirable for L to be of a substantial length. Placing a slot 1512 in the handle 1502 that the guide wire can enter and flex in and out of advantageously allows the physical length of the handle to be greater than A-A′.

Accordingly, in one embodiment the handle 1502 has a physical length that is longer than its effective length (as defined above). In one embodiment the deployment system 1500 has the proximal end of the guidewire lumen located distal to the proximal end of the handle, where the guidewire lumen is accessible to the physician, and the guidewire is coaxial with the delivery system through the distal portion of the handle.

With continued reference to FIGS. 16-17, the deployment system 1500 can also include a flush port 1514 coupled to a flush tubing 1515. Prior to tracking the deployment catheter 900 to the target location, it can be desirable to flush as much air as possible from the deployment system 1500. Air in the catheter has the potential to be released during the procedure, and if this occurs the air may cause an air embolism where the air in a blood vessel prevents oxygenated blood from reaching the adjacent tissues. In one arrangement, the deployment system 1500 is flushed with a saline solution and in one arrangement approximately 0.9% saline. In certain arrangements, the solution can also contain an anticoagulant such as heparin.

In one embodiment the procedure includes a final flushing step after the tip of the catheter is inserted into the introducer and before the catheter is advanced into the patient's vasculature or at least before the tip of the catheter is advanced past the great vessels. This procedure can flush out any residual air and traps it preferably in the introducer sheath or at least in the less critical peripheral tissues.

In one embodiment the flexible flush port tube 1515 is fluidly connected to the outer catheter 901 and a seal surface between the outer catheter 901 and inner catheter 904 prevent at least a portion and preferably a majority of the flush fluid from escaping proximally. Preferably an adequate amount of the flush fluid passes between the inner and outer catheters distal to the area where the implant is loaded within the catheter so that the fluid pushes out any air bubbles trapped near the implant. As will be described in more detail below, in the illustrated embodiment the end of the flush port 1514 that connects to the catheter moves axially relative to the body of the handle 1502 when the implant is unsheathed as the outer catheter is retracted. To accommodate the relative motion of the flush port 1514, in one embodiment the entire flush port tubing 1515 is able to move relative to the handle 1502 in the approximate direction of the axial motion used to unsheathe the implant. In some embodiments the flush port 1514 extends proximally, in other embodiments the flush port 1514 extends distally of the handle 1502. In another embodiment, the end of the flush port tube that is fluidly connected to the outer sheath moves with the outer sheath, but a portion of the flush port tube is fixed to the handle body. In such arrangements, sufficient length can be provided such that the outer sheath can be advanced to its most distal position relative to the handle body without excessively stretching the flush port tubing, or without stretching the flush port tubing enough to cause significant permanent deformation. Sufficient clearance can also be provided within the handle body such that the flush port tube can bend to accommodate the outer sheath in its most proximal position, without the tubing kinking. In another embodiment the flush port can be kinked in its most proximal position. In one embodiment the flush port is only operable with the outer sheath in the configuration where the device is introduced into the patient and the flush port becomes kinked in other configurations, this allows a smaller package in some embodiments.

As noted above, in one embodiment the flush port 1514 can be fluidly connected to the outer body of the handle and the outer sheath is fluidly connected to a component which moves with the outer catheter and fluidly seals to the handle body in at least one position of the outer sheath relative to the handle body. In one embodiment the flush port tubing is connected fluidly and mechanically to the handle body. When fluid is forced into the flush port a fluid chamber within the handle body is substantially filled with the flush fluid. In this embodiment the moving interface between the outer catheter and the inner catheter does not contain a seal at the handle end. The pressurized fluid within the chamber in the handle is able to flow between the inner catheter and the outer catheter thereby flushing air from the delivery system including the area where the implant is positioned. For this design the handle housing is advantageously relatively fluid tight and the internal volume can be minimized and shaped to minimize residual air pockets that may introduce air bubbles late in the flushing process.

In another embodiment the flushing step is performed by applying a vacuum to the flush port and submerging the distal end of the delivery system in fluid. This method has the advantage of flushing the device under vacuum so any small bubbles such as those trapped in fabric expand and are more likely to be flushed away.

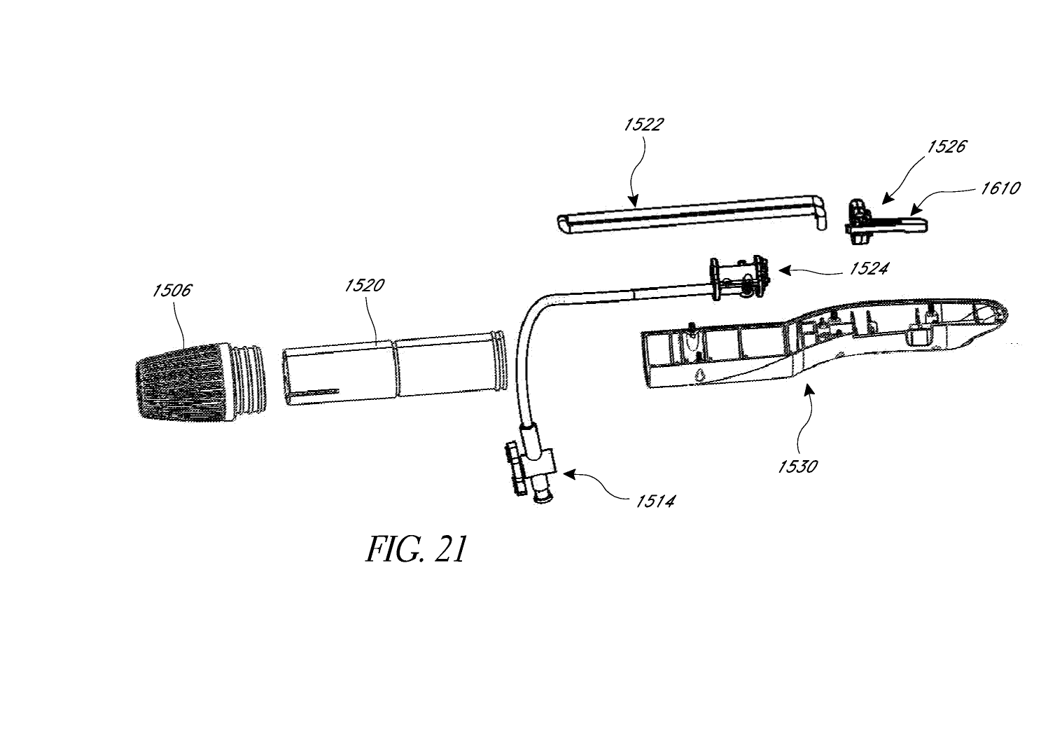

With reference to FIGS. 15-21, as noted above, distal end of the body includes an actuator 1506 in the form of a knob in the illustrated embodiment that can be rotated or twisted in order to retract the outer tubular member or outer sheath 901 while holding the inner tubular member 904 (not shown in FIG. 16A) stationary or stationary. With particular to FIG. 21, the mechanism for retracting the outer tubular member or outer sheath 901 can include the actuator 1506 or “knob” going forward, a screw member 1520, a track 1522, and a carriage 1524. As explained below, the knob 1506 can be used to rotate the screw member 1520 and can include ridges or knurling to aid gripping by the user. In other embodiments, the actuator can be in the form of a lever, dial or other mechanism configured to transfer rotational force.

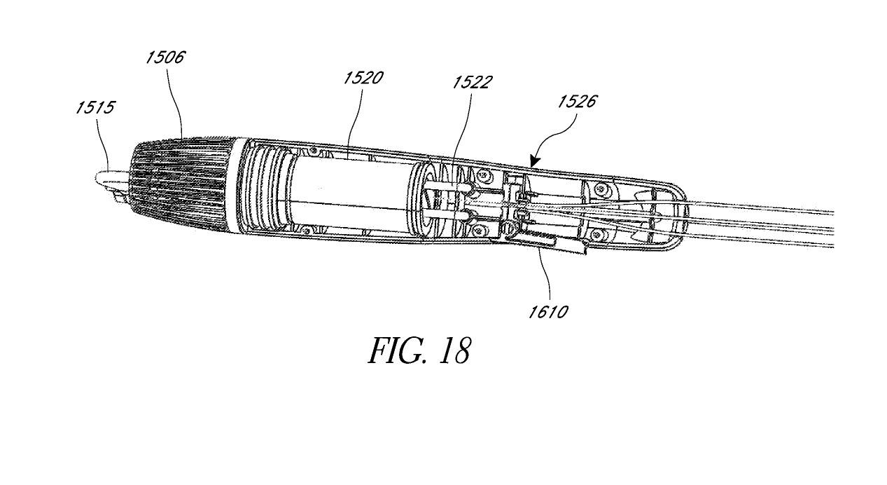

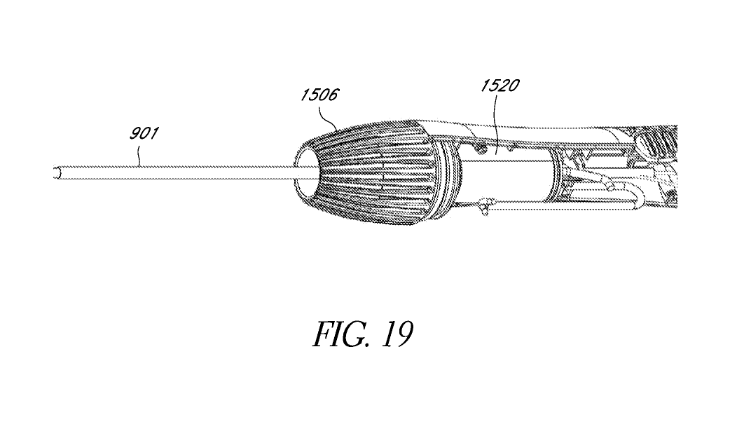

With continued reference to FIG. 21, the deployment mechanism 1500 can also include a lock mechanism 1526, which will be described in more detail below. Portions of the knob 1506, the screw member 1520, the track 1522, the carriage 1524 and the lock mechanism 1526 can be positioned within the handle 1502. To facilitate assembly, the handle 1502 can be formed from multiple components, such as, for example, in the illustrated embodiment an “top” half 1529 and a bottom half 1530 which can form “clam shell” halves of the handle 1502. The top and bottom halves 1529, 1530 can be connected together to define an internal cavity in which the aforementioned components (or portions thereof) can be positioned with the outer surface of the two halves forming the outer surface of the handle 1502. FIG. 18 illustrates the handle 1502 with the top half 1529 removed while FIG. 19 illustrates the bottom half removed. In modified arrangements, the handle 1502 can be formed for more or less components.

In the illustrated arrangement, a screw-mechanism can be used to deploy/unsheathe the device. In such an embodiment, the deployment mechanism 1500 can move the outer sheath 901 relative to the handle 1502 to retract the sheath 901 over the implant, while minimally (if at all) advancing the implant further. The illustrated arrangement can advantageously improve physician feel and comfort during the procedure, making the procedure easier, making training easier, and overall providing a more positive experience for the clinician and staff. Advantageously, the deployment mechanism 1500 would maintain both bioprosthesis functionality and feedback from the system during unsheathing, while yielding a solid feel, offering control, all while minimizing or reducing force.

Advantageously, the pitch of the screw mechanism will provide some mechanical assistance during unsheathing while also maintaining some feel for the operator. In one embodiment, unsheathing is a quick process, with less than 3.5 turns on the knob required to utilize the full unsheathing throw. As will be explained in detail below, in the illustrated arrangement of the screw mechanism, the “nut” (carriage) is placed inside of the “screw” (i.e., the screw member). This allows the design to be compact. In one arrangement, the “nut” or carriage can travel, at least partially, within the knob.

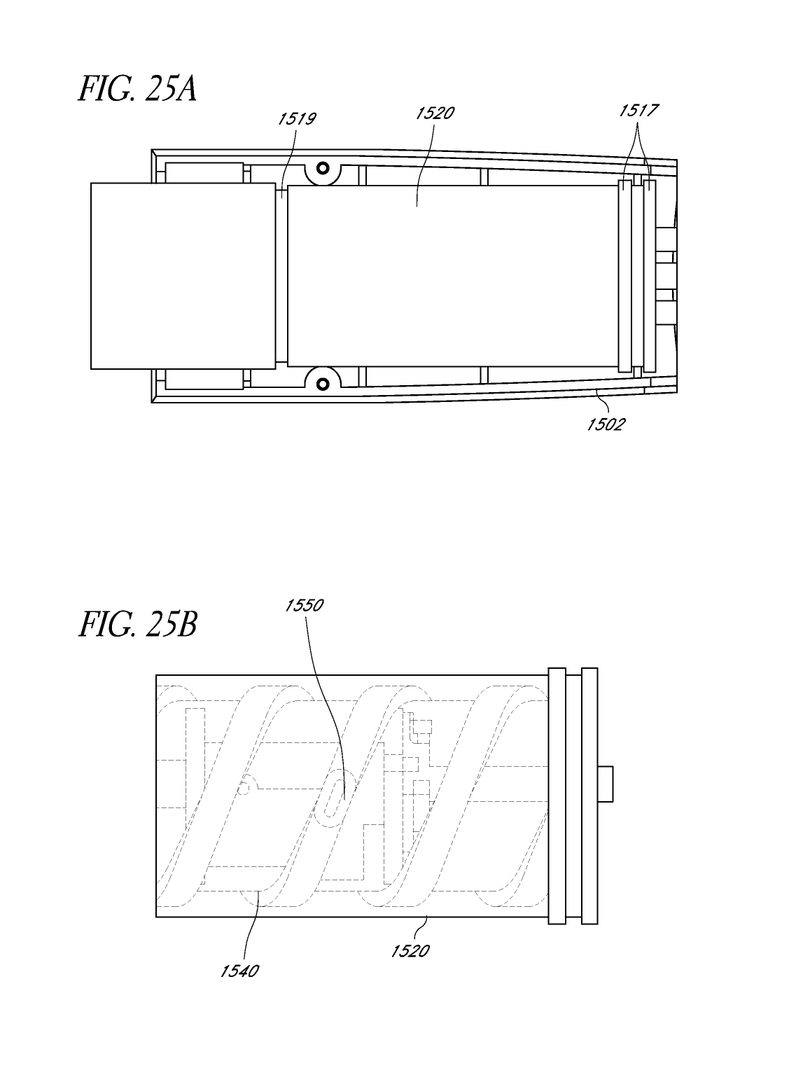

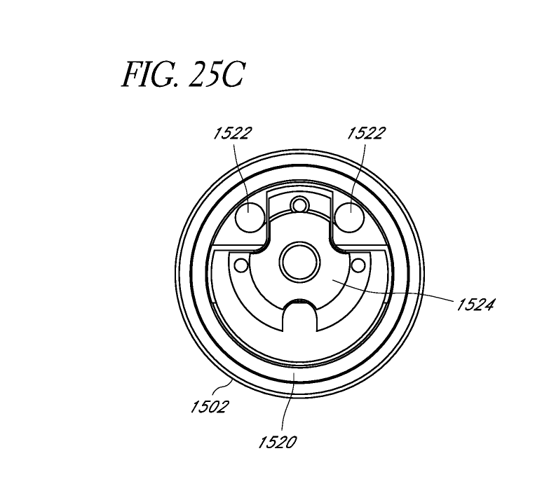

In one arrangement, the user rotates the knob 1506, preferably in the clockwise direction that in turn rotates the screw member 1520, which has internal threads 1534 (see FIG. 25B) and can have a cylindrical outer shape. The carriage (or nut) 1524 rides along the internal threads 1534 and can travel the length of the screw member 1520 or a portion thereof. The carriage 1524, in turn, can be coupled to the outer sheath 901 such that the outer sheath 901 is retracted as the carriage 1524 moves proximally within the screw member 1520. The inner tubular member 904 can be coupled to the handle 1502 (as described below) and can extend through the carriage 1524 and the outer sheath 901 such that movement of the carriage 1524 within the handle 1502 will cause relative movement of the outer sheath 901 with respect to the inner tubular member 904. The tubes 914, 916 can extend through the lumens in the inner tubular member 904 and out the slots and openings described above. As will be explained below, the carriage 1524 can also be coupled to the flush tubing 1515. The alignment member 1522 can extend within the screw member 1520 and can span the length (or a portion thereof) of the screw member 1520 and can keep the carriage 1524 in the proper orientation.

In one embodiment, the knob 1506 is an injection molded plastic, such as nylon and the OD is approximately 1.45″ and in one embodiment between 0.15″ and 14.5″ and the length accessible to the user is approximately 1.7″ and in one embodiment between 17″. Such dimensions and the dimensions for other components mentioned herein and below are provided as examples of an embodiment for understanding the arrangements of various components. Modified arrangements and embodiments can have different dimensions or ranges. The knob can span approximately an additional 0.55″ or in one embodiment 0.055 and 5.5″ into the handle 1502 to retain a rigid feel and distribute any load throughout the handle 1502. As shown in FIG. 16A, the tangible OD of the knob 1506 can comprise of several grooves or notches to add ergonomics and texture for the user interaction. A thin ring around the OD approximately 0.2″ wide in one arrangement can illustrate a marking illustrating which direction to turn the knob for unsheathing. In the illustrated embodiment, the marking comprises a series printed arrows.

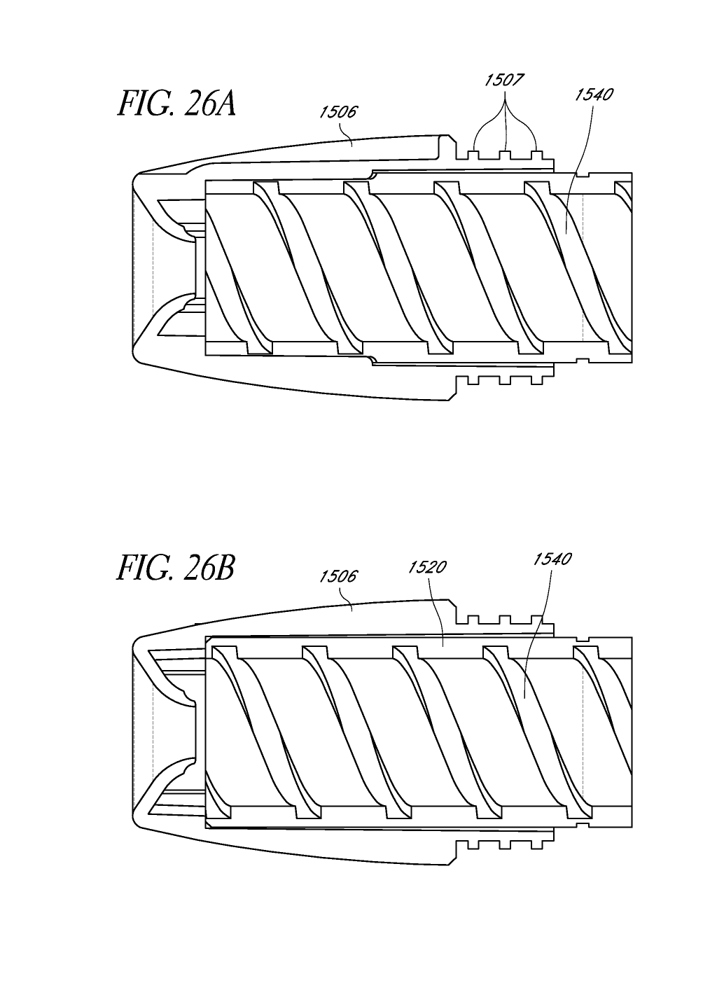

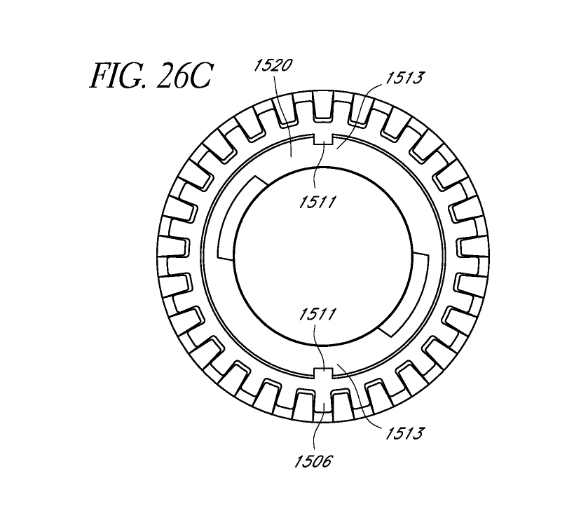

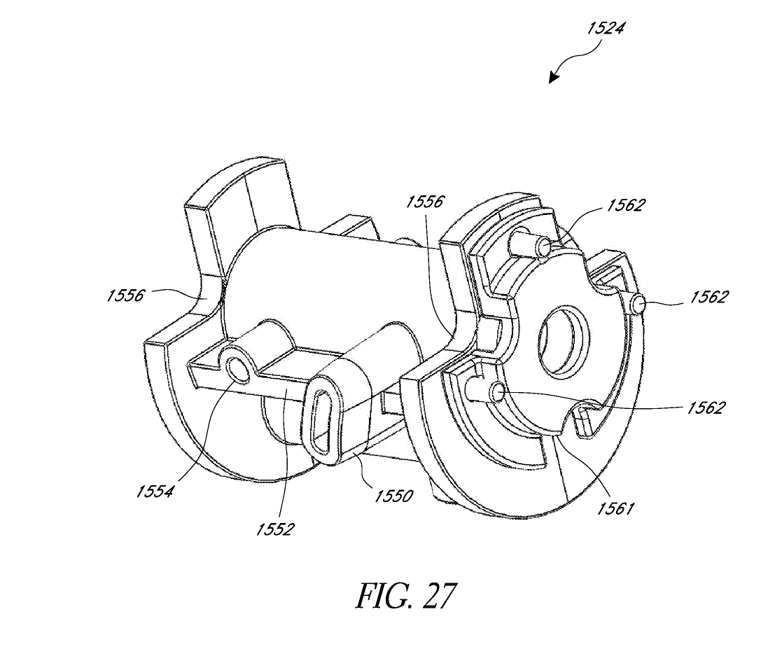

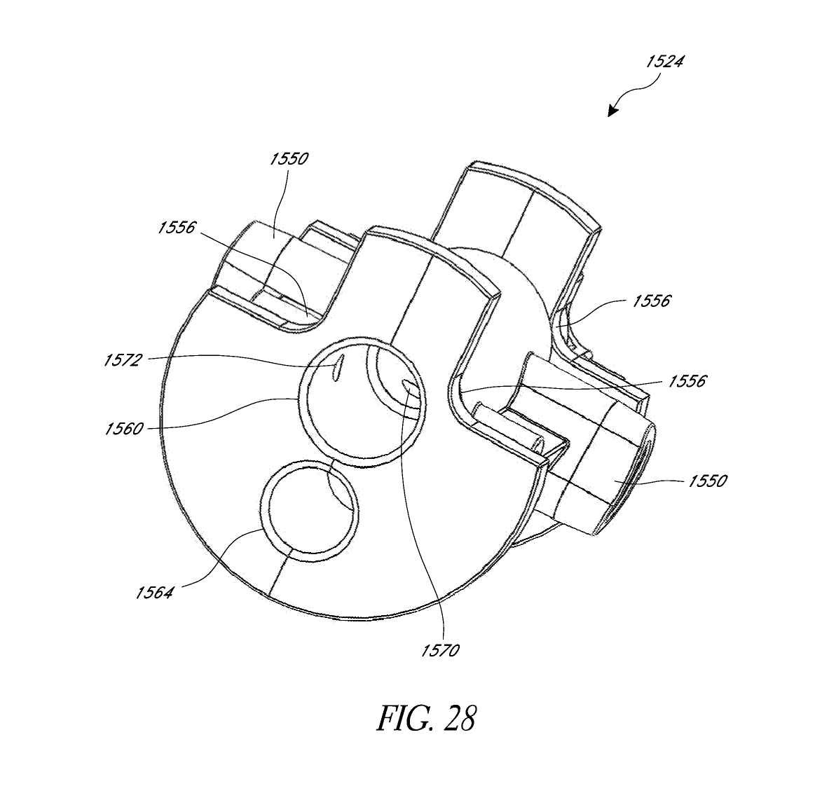

With reference to FIGS. 26A-C, a series of annular ribs 1507 can be positioned along the length of the knob positioned inside the handle shells/halves. The annular ribs 1507 can interact with corresponding annular grooves or ribs in the handle (see e.g., FIG. 19) to keep the knob 1506 oriented properly and limit axial movement of the knob 1506 with respect to the handle 1502. The ribs 1507 can be evenly spaced from the center of the length and in an embodiment are approximately 0.059″ wide and approximately 0.116″ apart and the OD of the ribs in an embodiment is approximately 1.25″ and the OD of the 0.55″ length is approximately 1.18″ in an arrangement. The illustrated embodiment includes 3 ribs 1507 with 3 corresponding grooves 1509 in the handle 1502 More or less ribs/grooves can be used in other arrangements. Also grooves and ribs can be interchanged and/or other structures can be used to allow rotation of the knob while restricting axial movement (e.g., various combinations of grooves, tabs, ridges, and ribs etc.).

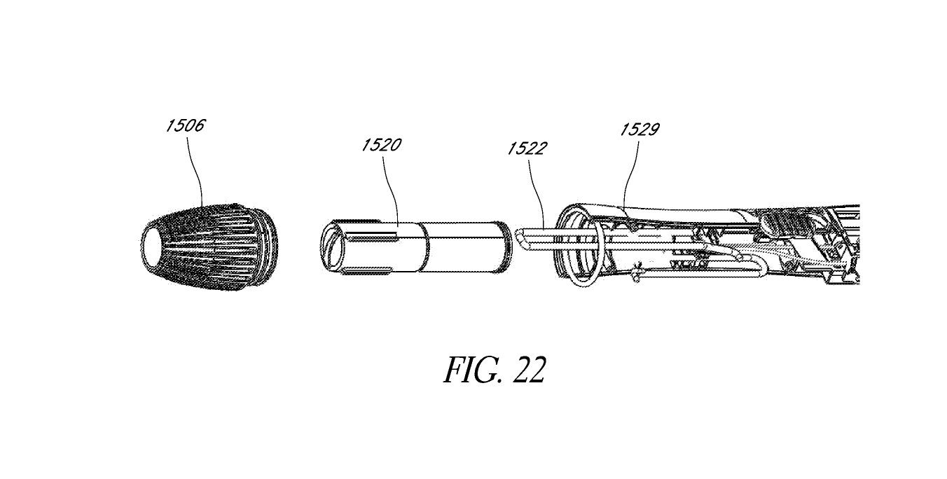

With continued reference to FIGS. 26A-C, in an embodiment, the distal ID of the knob is approximately 0.452″ and the proximal ID is approximately 1.08″. In the illustrated embodiment, this proximal ID contains a one or more of fins or ribs 1511 for structural support. In the illustrated arrangement, some ribs 1511 span the length of the ID. The ID and ribs 1511 of the knob 1502 can accept the distal OD of the screw member 1520, which possesses corresponding grooves 1513 configured to match the ribs 1511 of the knob 1506 such that rotation of the knob 1506 causes rotation of the screw member 1520. More or less ribs/grooves can be used in other arrangements. Also grooves and ribs can be interchanged and/or other structures can (e.g., various combinations of grooves, tabs, ridges, and ribs etc.). In certain arrangements, the knob 1606 and screw member 1520 (or portions thereof) can be formed a single piece or divided in to one or more connected components. FIG. 22 shows an arrangement in which the screw member has 4 ribs while FIG. 26 has a larger number of ribs.

In an embodiment, the screw member 1520 is an injection molded plastic, such as acetal, and in an embodiment is approximately 4.17″ long. In an embodiment, the largest OD if the member is approximately 1.096″ at the proximal ribbing that fits in the handle shells to hold the part co-linearly to the handle internals. In the illustrated embodiment (see e.g., FIGS. 25A-B), there are two of these ribs 1517 that are in an embodiment approximately 0.069″ wide and 0.071″ apart. In one arrangement, the OD of the proximal length is approximately 1.036″ and in the center of the part, there is a radial groove 1519 of approximately 0.064″ wide that reduces the OD to approximately 0.966″. In an embodiment, the distal OD can be tapered at approximately 1 degree for the duration of the length of the part. This tapered OD can possess the groove features that lock into the knob. They can be approximately 0.945″ long and at between 0.07″ and 0.1″ wide depending on location in an embodiment. The distance between the center of the part and the bottom of the groove can be approximately 0.477″ in an embodiment.

As shown in FIGS. 25B, 26A, 26B, the screw member 1520 can include the internal thread 1540. In the illustrated embodiment, the internal thread 1520 is a double-start thread with approximately a 1.0″ pitch. In one embodiment, the ID of the part is can be approximately 1 degree from both proximal and distal ends, meeting in the middle. The width of the thread groove can be approximately 0.138″ in one embodiment. The major diameter of the thread can be approximately 0.946″ and the threads can extend the entire length of the screw member in one embodiment. In one arrangement, the mold surface finish of the threads is a polish to increase lubricity. This can reduce the friction between the threads and the carriage/nut that rides in them through the length of the part.

With reference to FIGS. 27 and 28, in an embodiment, the carriage (or sometimes referred to as “nut”) 1524 is an injection molded plastic, such as polycarbonate. This part can be a clear material to allow for UV bonding process to allow for attachment of additional parts such as the outer sheath and hemostasis cap. The carriage 1524 can include two wings 1550, which in the illustrated arrangement are located 180 degrees apart. The wings 1550 are configured to ride in the internal threads 1540 of the screw member 1520. In certain arrangements, the carriage 1524 can have more or less wings 1550 arranged at different locations along the carriage 1524 and/or in different shapes. In an embodiment, the wings 1550 can be replaced and/or used in combination with grooves that interact with corresponding thread-like protrusions provided within the screw member 1520. The mold cavity of the wings 1550 can be a polished finish to minimize friction between the moving parts. In one embodiment, the wings 1550 can be angled at approximately 68 degrees to prevent cocking and un-desirable movement in the screw member 1520. In the illustrated arrangement, the wings 1550 are supported by ribs 1552 that run the length of the part. The ribs 1552 can include glue-ports 1554 to allow for bonding on the ID of the part. For example, in one arrangement, glue (or other adhesive) can be inserted through the glue port 1554 for wicking glue into the carriage 1524 to bond the outer sheath 901 extending through the through a through hole 1560 of the carriage 1524. The proximal and distal end of the part 1524 can be circular (approximately 0.76″ OD in one arrangement) in the illustrated embodiment with notches 1556 removed leaving a width of approximately 0.28″ in one embodiment. As explained below, the notches 1156 can be configured to ride along the alignment rod 1552. The OD of the wing profile can be approximately 0.897″ in one embodiment. The carriage 1524 can include several passages, through-holes, and blind holes for attaching components, bonding, and allowing parts to pass through it without impeding motion.

As shown in FIGS. 27 and 28, the through-hole 1560 can span the entire length of the part and starts with an ID of approximately 0.224″ at 1 degree draft at the distal end in one embodiment. In the illustrated arrangement, approximately 0.5″ though the part, the through hole 1560 steps down to an ID of approximately 0.175″ which tapers outwards towards the proximal end at about 1 degree in one embodiment. This ID can end in a counterbore of approximately 0.275″ ID which can receive a hemostasis seal in the illustrated arrangement. In one arraignment, this proximal half spans a length of approximately 0.495″, about 0.095″ of which is the counterbore. In the illustrated arrangement, on the proximal surface of the part, 3 pins 1562, approximately 90 degrees apart can be provided and can extend approximately 0.127″ and have an OD of approximately 0.055″ and are drafted at approximately 2 degrees. In other arrangements more or less pins or pins of different spacing can be provided. The proximal end of the carriage 1524 can also include a groove 1561 for receiving an O-ring or other sealing member. In an embodiment, a 70 durometer o-ring can be positioned on the groove 1561 to provide a hemostasis seal. In an embodiment, above the distal through hole 1560, there can be a distal blind hole 1564 of approximately 0.160″ ID and a length of approximately 0.465″ that steps down to a hold of about 0.06″ ID and about 0.137″ long. These holes can be drafted at approximately 1 degree. On the top of the part, there can be a blind hole and counterbore the spans from the top of the part into the through hole ID and is approximately 0.375″ deep in an embodiment. This hole can connect the aforementioned blind hole and through hole to allow for full flushing of the system. The counterbore of the hold on the top face of the part can be designed to be sealed with adhesive to create a cap.

With reference to FIG. 28, an opening 1570 can provide communication with the flush port. FIG. 28 also illustrates the opening 1772 for the adhesive port wick 1554 and a shoulder 1574, which can serve as a stop for the proximal end of the outer sheath 901.

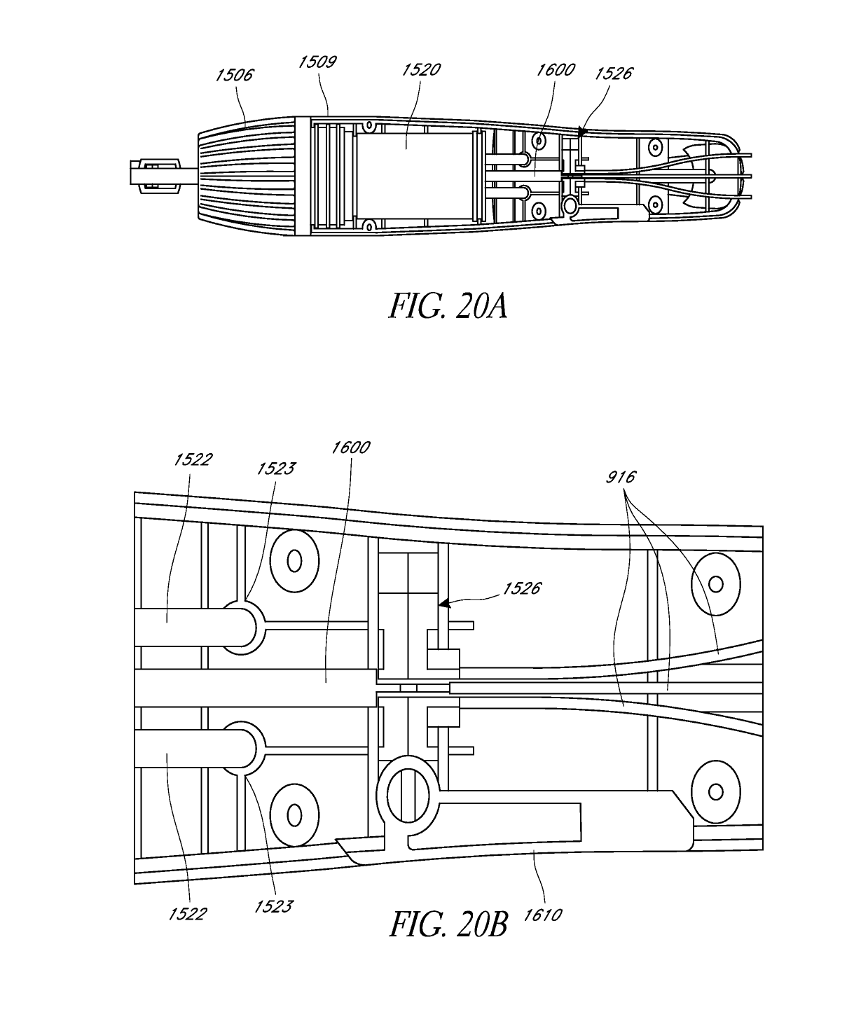

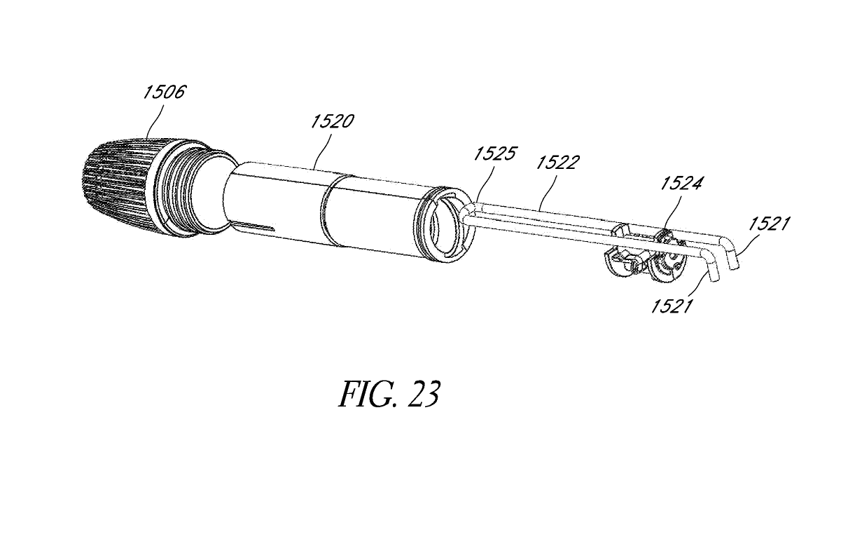

The alignment member 1522 is shown in FIGS. 21, 22 and 23. In the illustrated embodiment, the alignment member 1522 comprises a wire bent into a u-shape with two downturned ends 1521. The two downturned ends 1521 can be located at the proximal end of the alignment member 1522 and can be positioned within bosses 1523 (see FIG. 20B) formed in the lower half of the handle 1502 to constrain axial movement of the alignment member 1522 and to provide support. The distal bent end 1525 of the alignment member 1522 extends into the screw member 1520. As best seen in FIG. 23, the two legs of the alignment member 1522 can form “rails” which form a track along which the carriage 1524 can move within the screw member 1520. That is, in the illustrated arrangement, the carriage 1524 moves along the longitudinal axis of the of the alignment member 1522. In the illustrated arrangement, the alignment member 1522 sits above a centerline of the carriage 1524 as assembled into the screw member 1520. Modified arrangements can include a single rail (or more rails) and/or one or more track members that engage protrusions on the carriage 1524 or other configurations configure to prevent or limit rotation of the carriage 1524 within the screw member 1520.

Accordingly, when assembled, the carriage 1524 is positioned within the screw member 1520 and the wings 1550 engage the internal thread 1540 of the screw member. As the knob 1506 rotates the screw member 1520, the wings 1550 of the carriage 1524 move along the thread 1540 as rotation of the carriage 1524 is limited by the alignment member 1522. The result is the carriage 1524 moves axially within the screw member 1520 with rotation of the knob 1504. The proximal end of the outer sheath 901 can be coupled to the carriage 1524 such that movement of the carriage 1524 within the handle 1502 causes movement of the outer sheath 901 with respect to the handle 1502.

In the illustrated embodiment, the handle 1502 can be made of 2 sub-assemblies. In the first subassembly the proximal end of the outer sheath 901 is bonded to the carriage 1524 in the tapered socket 1560 of the carriage 1560. In one arrangement, the carriage is optically clear to verify that an adequate bond is formed. In an embodiment, the outer sheath 901 seats into carriage 1524 between 0.1 and 1 inches and in certain embodiments between 0.3 and 0.5 inches. The bond strength of this bond is in one arrangement is greater than 30, 50 or 60 lbs.

In the one embodiment, the screw member 1520 can include a mechanism to provide friction. Adding friction close to the user input can prevent a sloppy feel and minimize springback. In one embodiment the friction can created by a resistance o-ring.

In an embodiment knob 1506, engages the screw member 1520 with at least 2 bosses or ribs. In an embodiment, the screw member 1520 is located inside the knob 1506 to reduce the overall length of the delivery system 1500. In an embodiment at least 1, 2, or 3, inches of the screw member 1520 is located within the knob 1506. In one embodiment, the thread within the screw member 1520 and over which the carriage 1524 travels begins less than 0.3, 0.5 or 1 inches from the distal end of the handle assembly or the knob. In one embodiment, the carriage at least partially extends into the knob during motion of the carriage.

As shown in the figures, in the illustrated embodiment, the screw member has 2 radial grooves the engage ribs in the handle halves to limit axial movement during normal operation. In certain embodiments, more or less ribs or structures of different form can be used to limit movement. The screw member is preferably installed in handle halves to handle axial loads of at least 30, 50, or 1001 lbf without impact on function.

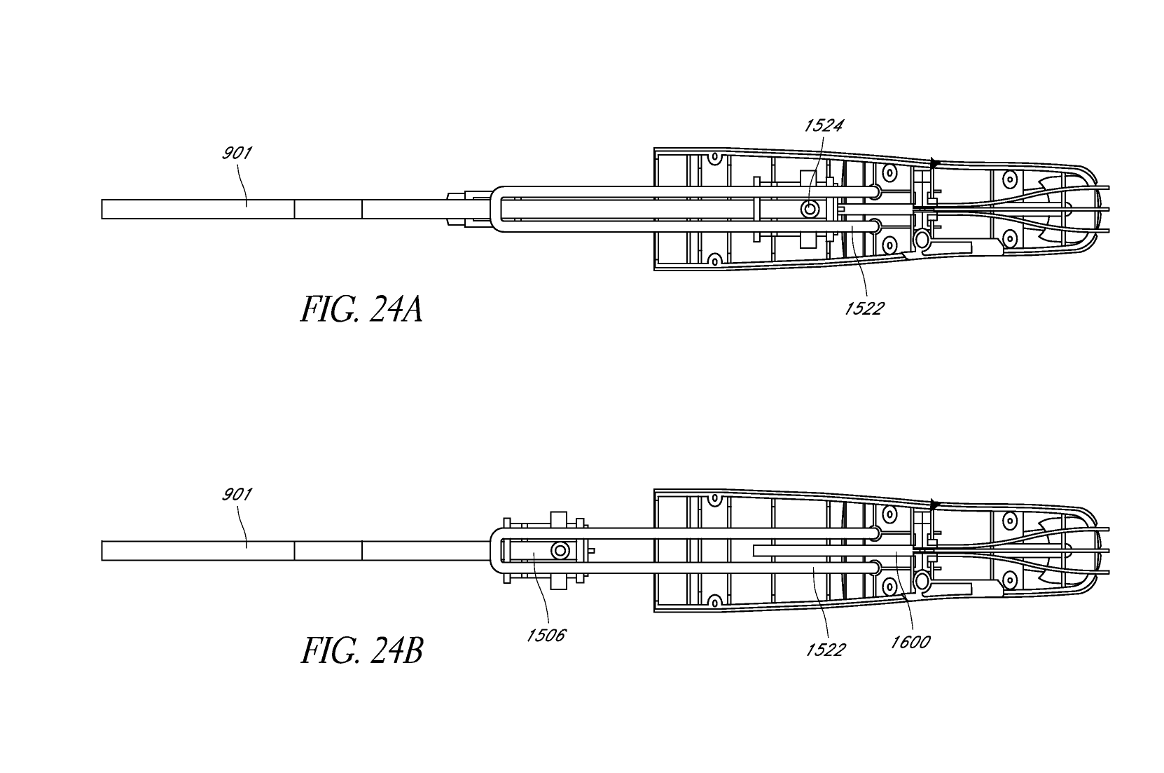

Accordingly, in the illustrated embodiment, the user can rotate the knob 1506, preferably in the clockwise direction, which, in turn, rotates the screw member 1520, which has the internal threads 1534 (see FIG. 25B). The carriage (or nut) 1524 rides on the internal threads 1534 and can travel the length of the screw member 1520 from a distal end to proximal end (or a portion thereof). The carriage 1524, in turn, can be coupled to the outer sheath 901 such that the outer sheath 901 is retracted as the carriage 1524 moves proximally within the screw member 1520. The carriage 1524 can also be coupled to the flush tubing 1515. The alignment member 1522 can extend within the screw member 1520 and can span the length of the screw member 1520 to keep the carriage 1524 in the proper orientation and to limit rotation of the carriage 1524 such that rotation of the screw member 1520 results in axial motion of the carriage 1524. FIGS. 24A and 24B illustrate the carriage 1524 in its most proximal position and its most distal position as it moves along the alignment member 1522. In the illustrated arrangement, both the outer sheath 901 and the flush tubing 1515 extending through an opening in the knob 1506 at the distal end of the handle (see e.g., FIGS. 26A and 26B which illustrate an opening in the knob 1506).

In the illustrated embodiment, the inner tubular member 904 extends through carriage 1524 and the outer heath 901. With reference to FIGS. 20A and 20B, the handle 1502 can be provided with a guide tube 1600 and the locking mechanism 1526. As shown in these figures, the guide tube 1600 can extend from the locking mechanism 1526. The inner tubular member 904 extends through the outer sheath 901, the carriage 1524 and the guide tube 1600 with a proximal end of the inner tubular member 904 positioned within the locking mechanism 1626. The position wires 916 inserted into a multi lumen tube prior and the guide wire lumen 194 extends from the locking mechanisms and through openings (or slots) at the proximal end of the handle. As explained below, the locking mechanism 1526 can be configured to clamp down on the inner tubular member 904 to limit axial movement between the handle 1502 and the inner member 904. In this manner, as the outer sheath 901 is proximally retracted as described above the inner member 904 can remain substantially stationary. In the illustrated arrangement, the position wires and guide wire lumen (and guide wire extending there through) can be axially moved within lumens of the inner tubular member 904 while it is clamped within the locking mechanism 1526.

During the usage of the device, the physician may need to release and remove a portion of the delivery system (e.g., the outer sheath 901). This locking mechanism 1526 can allow removal of a portion of the delivery system to make room for another device. As described below, this can involve disconnecting an inner member 904 of the delivery system. In some embodiments the disconnection and removal of the outer sheath 901 can allow a retrieval system or another catheter to be tracked over the inner member 904 of the catheter facilitating the retrieval of the implant through the introducer.

In illustrated embodiment this disconnection mechanism is the illustrated locking mechanism 1526 which can be in the form of a clamp that can fix the removable portion (e., outer sheath 901) of the delivery system until it is disengaged by the user to facilitate delivery system separation. In the illustrated embodiment, a collar that holds the delivery system together and actuation mechanism (e.g., a lever) facilitates disconnecting the delivery system.

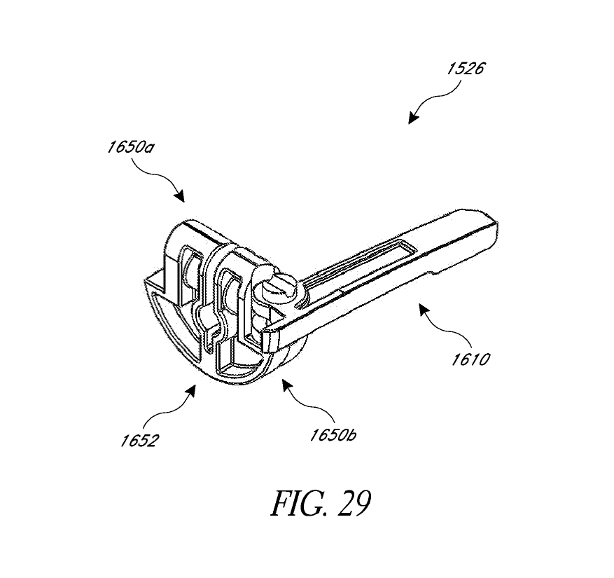

With reference to FIGS. 29-31, in the illustrated embodiment, the mechanism 1526 is a clamshell design with two halves 1650a, 1650b connected by a hinge 1652 that clamp together. A lever 1610 can be used to close the clamshell similar to a mechanism that secures a bicycle seat to a post. In the illustrated embodiment the clamp is a one piece design with one pinch point. In one embodiment the clamp can have features that index with corresponding features on the delivery system component to be released. In one embodiment the actuation mechanism utilizes an over center cam to squeeze the clamp together. In one embodiment the actuation mechanism utilizes a screw that is turned between 45 and 360 degrees to squeeze the clamp together. In one embodiment the actuation mechanism is actuated utilizing a lever 1610. In one embodiment the actuation mechanism utilizes a spring to pinch the clamp together. In one embodiment the clamp is made from a creep resistant material. In one embodiment the clamp is made from a fiber-reinforced polymer. In one embodiment the clamp is made from PEEK. In one embodiment the clamp is made from a metallic material. In one embodiment the clamp can withstand at least 2 lbs of force. The clamp can be designed to distribute the clamping force along a larger area of the inner tubular member 904, e.g., the axial length of the portion of the clamp that compresses against the inner tubular member 904 can be increased. Additionally and alternatively, the clamp can be tailored to accommodate the particular transverse cross-sectional diameter of the inner tubular member 904 that is being held within the clamp.

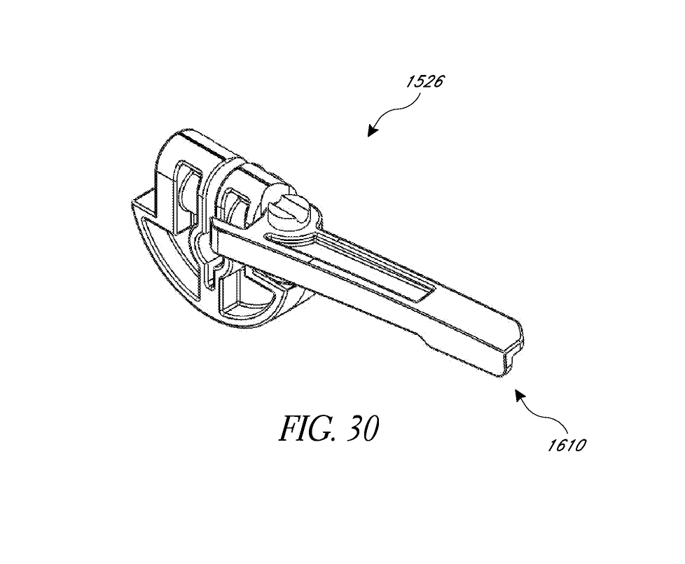

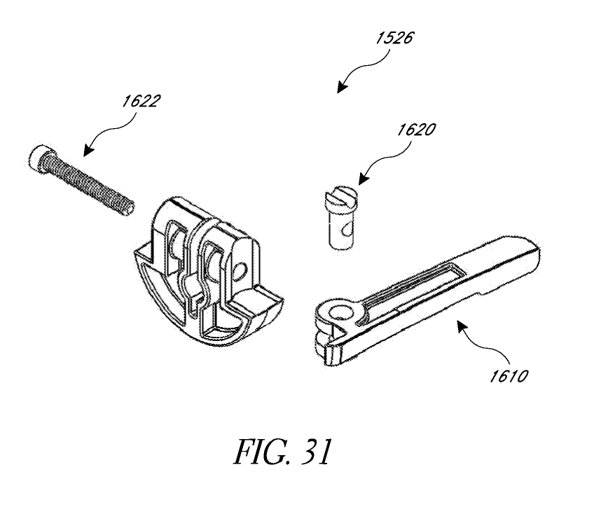

In one embodiment of use, the lever 1610 is moved from the locked position of FIG. 29 to the unlocked position of FIG. 30. This releases the clamping force exerted on the inner tubular member 904. Tabs at stem region 917 of the position wires (see FIG. 5B) can be removed. The handle 1502 can then be retracted over the inner tubular member 904, the guide wire lumen and the position wires. In this manner, the outer sheath 901 can be removed leaving the inner tubular member 904, the guide wire lumen and the position wires positioned within the patient. As shown FIG. 31, a pivot pin can be used to secure the level 1610 to the mechanism 1526 and a screw can be provided for securing the mechanism 1526 within the handle.

In an embodiment of use, a retrieval system can then be inserted over the inner tubular member 904. The retrieval system can be designed to remove the implant from the body through, for example, an introducer catheter if the implant size or its final position relative to the native annulus is not optimal. The device can be removed from the patient using the retrieval system at any point in the procedure prior to the exchange of the polymer. In one embodiment, the retrieval includes a basket into which the implant is retracted. The retrieval basket can then be retracted into the introducer catheter.

The above-describe methods generally describes an embodiment for the replacement of the aortic valve. However, similar or modified methods could be used to replace the pulmonary valve or the mitral or tricuspid valves. For example, the pulmonary valve could be accessed through the venous system, either through the femoral vein or the jugular vein. The mitral valve could be accessed through the venous system as described above and then trans-septaly accessing the left atrium from the right atrium. Alternatively, the mitral valve could be accessed through the arterial system as described for the aortic valve, additionally the catheter can be used to pass through the aortic valve and then back up to the mitral valve. Additional description of mitral valve and pulmonary valve replacement can be found in U.S. Patent Publication No. 2009/0088836 to Bishop et al.

The various methods and techniques described above provide a number of ways to carry out the embodiments described herein. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments disclosed herein. Similarly, the various features and/or steps discussed above, as well as other known equivalents for each such feature or step, can be mixed and matched by one of ordinary skill in this art to perform combinations, sub-combinations and methods in accordance with principles described herein. Additionally, the methods which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein

What is claimed is:

1. A delivery system for delivering a cardiovascular prosthetic implant, the delivery system comprising:
a delivery catheter comprising an outer sheath with a proximal end portion and an inner sheath extending at least partially through the outer sheath, the inner sheath having a proximal end portion;
a handle at a proximal end portion of the delivery catheter;
a screw member positioned at least partially within the handle, the screw member configured for rotation about an axis within the handle, the screw member including an internal thread;
a carriage positioned within the screw member and engaging the internal thread, the carriage coupled to the proximal end portion of the outer sheath; and
an alignment member within the screw member, the alignment member contacting the carriage to limit rotation of the carriage about the axis as the screw member is rotated;
wherein rotation of the screw member in a first direction about the axis causes the carriage to move in a first longitudinal direction within the screw member causing the outer sheath to move in the first longitudinal direction relative to the handle.

2. The delivery system of claim 1, comprising an actuator for rotating the screw member with respect to the handle.

3. The delivery system of claim 1, wherein the actuator comprises a knob and the carriage is at least partially positioned within the knob as the carriage moves from a first position to second position.

4. The delivery system of claim 1, wherein the inner sheath extends through the proximal end portion of the outer sheath and the carriage and a proximal end portion of the inner sheath is coupled to the handle.

5. The delivery system of claim 4, further comprising a coupling mechanism positioned within the handle, the coupling mechanism configured to releasably couple the proximal end portion of the inner sheath to the handle.

6. The delivery system of claim 1 further comprising a cardiovascular prosthetic implant at a distal end of the delivery catheter.

7. The delivery system of claim 6, wherein the cardiovascular prosthetic implant comprises an inflatable cuff and a tissue valve.

8. The delivery system of claim 1, further comprising at least one link between the delivery catheter and the cardiovascular prosthetic implant.

9. A method of positioning a prosthetic implant within a heart, the method comprising:
advancing a delivery catheter comprising a prosthetic valve positioned within an outer sheath into a patient's vascular system;
translumenally advancing the prosthetic valve to a position proximate a native valve of the heart; and
deploying the prosthetic valve by retracting the outer sheath by rotating a screw member positioned within a handle of the delivery catheter to cause a carriage coupled to the outer sheath and positioned within the screw member to linearly retract within the screw cylinder as the screw member is rotated.

10. The method of claim 9, comprising grasping and rotating an actuator carried by the handle to rotate the screw member.

11. The method of claim 10, wherein the carriage is positioned at least partially within the actuator as the outer sheath is retracted.

12. The method of claim 9, further comprising releasing a coupling mechanism positioned within the handle to uncouple an inner member extending through the outer sheath from the handle.

13. A handle for a catheter system that includes a first member and a second member; the handle comprising:
a screw member positioned within the handle and configured for rotation about an axis, the screw member including an internal thread;
a carriage positioned within the screw member, the carriage engaging the internal thread and being coupled to the first member; and
an alignment member extending within the screw member to limit rotation of the carriage about the axis as the screw member is rotated;
wherein rotation of the screw member in a first direction about the axis causes the carriage to move in a first longitudinal direction within the screw member causing the first member to move in the first longitudinal direction relative to the handle.

14. The delivery system of claim 1, further comprising an actuator for grasping and rotating the screw member, wherein the carriage is positioned at least partially within the screw member as it moves from a first position to a second position.

15. A method of retracting an outer sheath relative to an inner sheath of a catheter comprising:
rotating a screw member positioned within a handle of the delivery catheter to cause a carriage coupled to the outer sheath and positioned within the screw member to linearly retract within the screw cylinder as the screw member is rotated.
